(19) United States
(12) Patent Application Publication
(10) Pub. No.: US 2020/0306303 A1
(43) Pub. Date: Oct. 1, 2020

(54) METHODS AND COMPOSITIONS ENHANCING SURVIVAL AND FUNCTIONALITY OF ANTI-TUMOR AND ANTI-VIRAL T CELLS

(71) Applicants: Chiou Hwa YUH, Rosemead, CA (US); National Health Research Institutes, Zhunan Township, Miaoli County (TW)

(72) Inventors: Li-Rung HUANG, Zhunan Township, Miaoli County (TW); Shu-Ching HSU, Zhunan Township, Miaoli County (TW)

(21) Appl. No.: 16/652,350

(22) PCT Filed: Oct. 1, 2018
(86) PCT No.: PCT/US2018/053692

§ 371 Date: Mar. 30, 2020

Related U.S. Application Data
(60) Provisional application No. 62/565,820, filed on Sep. 29, 2017.

(51) Int. Cl. A61K 35/17; C12N 9/12; C07K 14/705; C12N 15/86; A61P 35/00

(52) U.S. Cl. CPC A61K 35/17; C12N 9/12; C12Y 207/11001; C07K 2319/033; C12N 15/86; A61P 35/00; C07K 14/705

(57) ABSTRACT

The present invention relates to a method able to enhance survival and functionality of anti-tumor or anti-viral immune cells through overexpression of Akt molecules in the cells. Akt signaling prevented the expression of immune checkpoints and therefore rescued antigen-specific cytotoxic T lymphocytes from exhaustion in immunosuppressive microenvironment. This present invention also demostrated that AKT genes have the potential to be utilized in T-cell engineering of adoptive T-cell therapy for treatment of chronic viral infection and malignancies

### CROSS-REFERENCE TO RELATED APPLICATION

This application claims priority to U.S. Provisional Application No. 62/565,820, filed on SEP 29, 2017, the entire content of which is hereby incorporated by reference herein in its entirety.

### BACKGROUND OF THE INVENTION

### Technical Field of the Invention

The present invention relates to adoptive cell therapy using Akt-overexpressing immune cells. More specifically, the Akt-overexpressing immune cells can be utilized for treatment of viral infection and malignancies in immunosuppressive microenvironment.

### Background

Adoptive cell therapy (ACT) utilizing gene engineering to introduce antigen specificity or to enhance effector functions or survival of immune cells is feasible and high clinical values for treatment of chronic infections or malignancies since virus- or tumor-specific immune cell response is usually impaired or missing in patients with most of these chronic diseases.

However, during chronic viral infections or malignancies, there are usually monoclonal T cell response detected and most of the antigen-specific T cells undergo exhaustion or apoptosis rapidly after activation. It is often observed that the virus or tumor-specific cytotoxicity T lymphocytes (CTLs) undergo T-cell exhaustion due to persistent T-cell receptor (TCR) signaling and lack of suitable co-stimulation. T cell exhaustion features the gradual loss of proliferative capability and cytokine production, impaired cytotoxicity, surface expression of various immune checkpoints and increase of apoptotic rate[1, 2].

Immune checkpoints e.g. PD-1 and CTLA-4 are molecules up-regulated on T cells in response to TCR signaling to modulate the extent of T-cell activation and are highly expressed on exhausted T cells. It has been shown in several studies that signaling through immune checkpoints on T cells can impair metabolic reprogramming during T-cell activation and differentiation[3-6].

The molecular pathways by which most of the immune checkpoints signal remain poorly understood except that PP2A and SHP2 activated by PD-1 and CTLA-4 signaling, respectively, can suppress Akt activation of T cells upon TCR stimulation, being revealed[7].

Akt is shown to have a great influence on T-cell growth, proliferation, and survival and also demonstrated to be a signal integrator for T-cell differentiation through regulation of Foxo, mTOR and Wnt/β-catenin pathways[8-11]. During chronic LCMV infection, the activation of Akt and mTOR signaling in CTLs is impaired, which results in T-cell exhaustion through PD-1 signaling in virus-specific CTLs[12].

Therefore, the present invention demonstrates that reinforcement of Akt/mTOR pathway in anti-viral or anti-tumor CTLs may rescue them from T cell exhaustion and has the potential to be further applied on recombinant TCR technology or chimeric antigen receptor (CAR) technology [13] to enhance the survival and effector functions of engineered T cells for treatment of patients with malignancy or chronic viral infection.

### SUMMARY OF INVENTION

The present invention provides a method able to enhance survival and functionality of anti-tumor or anti-viral T cells through overexpression of Akt molecules in CTLs. The Akt-overexpressing CTLs are shown to have high proliferative capability and superior effector functions during encounter with the antigen in the liver, which suggests that the Akt molecules can help the CTLs to overcome T-cell exhaustion in the inhibitory microenvironment. We further show expression of Akt molecules can facilitate anti-viral and anti-tumor CTL responses e.g. proliferation, cytokine production and cytotoxicity. Moreover, it enables the CTLs resistance to proliferative arrest induced by MDSCs. the expression of constitutively active Akt molecules enable T cells to gain the privilege to survive and to kill in the tolerogenic liver or tumor microenvironments. The active Akt molecules only when in combination with TCR signaling can trigger massive proliferative response of CTLs and therefore are safe to be applied to T-cell engineering of CTLs.

In one embodiment, this present invention demonstrates that the myristorylated Akt molecules are able to anchor on cell membrane and can be phosphorylated. After being adoptive transfer into the recipient mice, Akt1- and Akt2-CTL populations expand vigorously in the liver and the spleen. It indicates overexpression of Akt is related to intrahepatic survival or secondary expansion of CTLs in response to antigen stimulation.

T cell exhaustion features surface expression of various immune checkpoints. The immune checkpoint blockade can rescue T cell exhaustion of CTLs and further enhance the anti-tumor responses. In another embodiment, this present invention demonstrated that Akt signaling prevents the expression of immune checkpoints, especially LAG-3 and TIGIT on HBV-specific CTLs.

In some embodiments, this present invention demonstrates that Akt1/2-engineered CTLs clear intrahepatic viral infections efficiently in two different models and persist and provide protective memory immunity in the recovered individuals.

In some embodiments, Akt2-engineered CTLs are able to eradicate established liver cancers in an oncogene-induced HCC mouse model. AKT1 and AKT2 genes can be utilized in T-cell engineering of adoptive T-cell therapy for treatment of hepatic chronic viral infection and malignancies since Akt signaling is able to reverse T-cell exhaustion of CTLs in immunosuppressive microenvironment.

### BRIEF DESCRIPTION OF THE DRAWINGS

FIGS. 1A-1O depict the HBV-specific CTLs undergo T-cell exhaustion after adoptive transfer into HBV carrier mice. (A) Kinetics of serum HBeAg of AdHBV-infected mice receiving adoptive transfer of 2×10⁵ HBc₉₃₋₁₀₀-specific CTLs. Gating (B) and quantification (C) of CD45.1⁺ transferred CTLs in the liver and the spleen of HBV carrier mice at indicated time points post adoptive transfer into AdHBV-infected mice. 5×10⁵ in-vitro activated HBc₉₃₋₁₀₀ CD8⁺ T cells are adoptively transferred into CD45.2⁺ recipient mice infected with AdHBV. Histograms show the expression of PD-1 (D, H, L), TIM-3 (E, I, M) and LAG-3 (F, J, N), on transferred CTLs in the liver and in the spleen of AdHBV-infected mice from day 3, 7 and 14 post adoptive transfer. The isotype control staining is shown in solid gray histogram whereas the specific staining is shown in open histogram. Mean fluorescence intensity (MFI) of PD-1, TIM-3 and LAG-3 staining on endogenous CD8⁺ T cells and on adoptively transferred CD45.1+CD8⁺ T cells (gating as shown in B) in the liver and in the spleen of AdHBV-infected mice from day 3(G), 7 (K) and 14 (O) post adoptive transfer of 5×10⁵ HBc₉₃₋₁₀₀-specific CTLs. **P<0.01 and ***P<0.001 (unpaired Student's t-test).

FIGS. 2A-2F depict the regulation of intrahepatic CTL expansion by different Akt isoforms. (A) Schematic representation of MSCV retroviral constructs used for T-cell engineering contain 5′ and 3′long terminal repeats (LTR), P2A linker peptide sequence (2A) (SEQ ID NO: 10), CD90.1 gene and woodchuck hepatitis virus posttranscriptional regulatory element (WPRE). In pMSCV-mAkt1/Akt2/Akt3-2A-CD90.1 plasmid, src myristoylation sequence (myr) (SEQ ID NO: 8) and mouse AKT1 (SEQ ID NO: 2). AKT2 (SEQ ID NO: 4) or AKT3 (SEQ ID NO: 6) gene are placed upstream of 2A sequence. (B) Transduction efficiency of in vitro-activated CD45.1⁺ OT-I cells transduced with retroviruses carrying 2A-CD90.1, mAkt1-2A-CD90.1, mAkt2-2A-CD90.1 and mAkt3-2A-CD90.1, respectively or mock. At day 2 after transduction, the surface expression of CD90.1 as a marker for successful transduction is detected by flow cytometric analysis. (C) Western blot for detection of phospho-Akt, total Akt, β-actin and phospho-S6 proteins in the cell lysate of ctrl, Akt1, Akt2 and Akt3-transduced CD8⁺ T cells. (D) Quantification of transferred CTLs in the liver and spleen of the mice with intrahepatic expression of the cognate antigen. 1×10⁵ transduced OT-I CTLs are adoptively transferred into recipient mice receiving hydrodynamic injection (HDI) of a plasmid encoding ovalbumin and luciferase under the control of albumin promoter one day before adoptive transfer. The liver-associated lymphocytes and splenocytes are isolated at day 7 after adoptive transfer and subjected to flow cytometric analysis of the percentage and the number of the transferred CTLs. Kinetics of accumulation of transferred CTLs in the liver (E) and spleen (F) of the mice with intrahepatic expression of the cognate antigen (ovalbumin). 1×10⁵ transduced OT-I CTLs are adoptively transferred into recipient mice receiving HDI of a plasmid encoding ovalbumin and luciferase under the control of albumin promoter one day before adoptive transfer. The liver-associated lymphocytes and splenocytes are isolated at day 3, 7 and 15 and subjected to flow cytometric analysis of the percentage and the number of the transferred CTLs. *P<0.05, **P<0.01 and ***P<0.001 (unpaired Student's t-test)

FIGS. 3A-3B depict the local expansion of Akt2-engrafted OT-I CTLs. (A) Kinetics of hepatic in vivo bioluminescence in mice receiving HDI of a plasmid encoding OVA under the control of albumin promoter or a ctrl vector (ctrl) one day before adoptive transfer of 2A-luc-engineered (ctrl) OT-I or mAkt2-2A-luc-engineered (Akt2) OT-I cells. The bioluminescence of individual mouse is monitored at day 1, 4, 8, 10, 12, 15, 18 and 25 after adoptive transfer and plotted in (B).

FIGS. 4A-4T depict the Akt-engineered HBc₉₃₋₁₀₀-specific CTLs overcame T-cell exhaustion in the liver. Histograms of expression of PD-1 (A), TIGIT (B) and LAG-3 (C) on Akt1-CD90.1- or CD90.1-engineered (ctrl) CTLs before adoptive transfer. The isotype control staining is shown in solid gray histogram whereas the specific staining is shown in open histogram. (D) Mean fluorescence intensity (MFI) of the staining results from A-C is shown in bar graph. (E) PD-1, (F) TIGIT and (G) LAG-3 on CD90.1-engineered (ctrl) CTLs and Akt1-CD90.1-engineered CTLs after 24-hours re-stimulation with anti-CD3/CD28 beads. The isotype control staining is shown in solid gray histogram whereas the specific staining is shown in open histogram. (H) MFI of the staining results from E-G is shown in bar graph. 5×10⁵ Akt1-CD90.1- or CD90.1-engineered (ctrl) are adoptively transferred into CD45.2⁺ recipient mice being infected with AdHBV. The liver-associated lymphocytes and splenocytes are isolated at day 6 or day 19 post adoptive transfer and subjected to flow cytometric analysis of the expression of immune checkpoints by the transferred CTLs. CD8⁺ CD45.1⁺ cells are gated and defined as transferred CTLs. Expression of immune checkpoints, PD-1 (I, J), TIM-3 (M, N) and LAG3 (Q, R), on transferred CTLs from day 6 post adoptive transfer. Expression of immune checkpoints, PD-1 (K, L), TIM-3 (O, P) and LAG3 (S, T), on transferred CTLs from day 19 post adoptive transfer. The isotype control staining is shown in solid gray histogram whereas the specific staining is shown in open histogram. MFI of the staining results are shown in J, L, N, P, R and T. (n=3 per group). *P<0.05, **P<0.01 and ***P<0.001 (unpaired Student's t-test)

FIGS. 5A-5H depict the influence of Akt signaling in the expression of immune checkpoints in vitro. Histograms of expression of (A) PD-1, (B) TIGIT and (C) LAG-3 on CD90.1-engineered (ctrl) CTLs, Akt1-CD90.1- and Akt2-CD90.1 CTLs after 3-days stimulation with anti-CD3/CD28 beads. The isotype control staining is shown in solid gray histogram whereas the specific staining is shown in open histogram. (D) MFI of the staining results from A-C is shown in bar graph. Histograms of expression of (E) PD-1, (F) TIGIT and (G) LAG-3 on CD90.1-engineered (ctrl) CTLs and Akt2-CD90.1-engineered CTLs after 24-hours re-stimulation with anti-CD3/CD28 beads. The isotype control staining is shown in solid gray histogram whereas the specific staining is shown in open histogram. (H) MFI of the staining results from E-G is shown in bar graph. *P<0.05, **P<0.01 and ***P<0.001 (unpaired Student's t-test)

FIGS. 6A-6F depict the Akt2-engineered HBc₉₃₋₁₀₀-specific CTLs prevent T-cell exhaustion in a persistent HBV mouse model. 2×10⁶ Akt2-CD90.1- or CD90.1-engineered (ctrl) is adoptively transferred into CD45.2+ recipient mice infected with AdHBV. The liver-associated lymphocytes and splenocytes are isolated at day 19 post adoptive transfer and subjected to flow cytometric analysis of the expression levels of immune checkpoints. Histograms of expression of PD-1 (A), TIM-3 (C) and TIGIT (E) on transferred CTLs in the spleen or liver of recipient mice at day 19 post adoptive transfer. The isotype control staining is shown in solid gray histogram whereas the specific staining is shown in open histogram. MFI of the staining results is shown in B, D and F. (n=3 per group). *P<0.05, **P<0.01 and ***P<0.001 (unpaired Student's t-test)

FIGS. 7A-7O depict the Akt-engineered HBc₉₃₋₁₀₀-specific CTLs developed protective immunity against HBV in a persistent HBV mouse model. Gating (A) and quantification (B, C) of CD45.1⁺ transferred CTLs in the liver and the spleen of HBV carrier mice at day 6 (B) or day 19 (C) post adoptive transfer into AdHBV-infected mice. 5×10⁵ Akt1-CD90.1- or CD90.1-engineered (ctrl) are adoptively transferred into CD45.2⁺ recipient mice being infected with AdHBV 2.5 months ago. The liver-associated lymphocytes and splenocytes are isolated at day 6 or day 19 post adoptive transfer and subjected to flow cytometric analysis of the percentage and the number of the transferred CTLs. CD8⁺ CD45.1⁺ cells are gated and defined as transferred CTLs. (D) Kinetics of serum HBeAg of recipient mice as in C. (E) Kinetics of serum ALT of recipient mice as in C. (F) Hematoxylin-and-eosin staining of the liver tissues from B. Immunohistochemical analysis of HBcAg (G), cleaved caspase 3 (H), Gr-1 (I) and CD45.1 (J) in the liver from B. (K) Hematoxylin-and-eosin staining of the liver tissues from C. Immunohistochemical analysis of HBcAg (L), cleaved caspase 3 (M), Gr-1 (N) and CD45.1 (O) in the liver from C. (n=3-4 per group). *P<0.05, **P<0.01 and ***P<0.001 (unpaired Student's t-test). Scale bars, 100 or 40 μm.

FIGS. 8A-8D depict the Akt2-engineered HBc₉₃₋₁₀₀-specific CTLs develope protective immunity against HBV in a persistent HBV mouse model. Gating (A) and quantification (B) of CD45.1⁺ transferred CTLs in the liver and the spleen of HBV carrier mice at day 19 post adoptive transfer into AdHBV-infected mice. 2×10⁶ Akt2-CD90.1- or CD90.1-engineered (ctrl) HBc₉₃₋₁₀₀-specific CTLs are adoptively transferred into CD45.2⁺ recipient mice infected with AdHBV. The liver-associated lymphocytes and splenocytes are isolated day 19 post adoptive transfer and subjected to flow cytometric analysis of the percentage and the number of the transferred CTLs. CD8⁺ CD45.1⁺ cells are gated and defined as transferred CTLs. (C) Kinetics of serum ALT of recipient mice as in B. (D) Kinetics of serum HBeAg of recipient mice as in B. *P<0.05, **P<0.01 and ***P<0.001 (unpaired Student's t-test)

FIGS. 9A-9D depict the cytokine production in HBV-specific CTLs after adoptive transfer into HBV carrier mice. (A) Zebra plots of intracellular expression of IFN-γ and TNF-α in adoptively transferred HBV-specific CTLs. 5×10⁵ Akt1-CD90.1- or CD90.1-engineered (ctrl) HBc₉₃₋₁₀₀-specific CTLs are adoptively transferred into CD45.2⁺ recipient mice infected with AdHBV. The liver-associated lymphocytes and splenocytes are isolated at day 19 post adoptive transfer and subjected to re-stimulation with HBc₉₃₋₁₀₀ peptides for 6 hours, which is followed by staining of surface markers and intracellular cytokines and flow cytometric analysis of the percentage of the cytokine-secreting CTLs. CD8⁺ CD45.1⁺ cells are gated and defined as transferred CTLs. (B) Bar graph of the percentage of IFN-γ-secreting CTLs (SP) and the percentage of CTLs secreting both IFN-γ and TNF-α (DP). (C) Zebra plots of intracellular expression of IFN-γ and TNF-α in adoptively transferred HBV-specific CTLs. 5×10⁵ Akt2-CD90.1- or CD90.1-engineered (ctrl) HBc₉₃₋₁₀₀-specific CTLs are adoptively transferred into CD45.2⁺ recipient mice infected with AdHBV. The liver-associated lymphocytes and splenocytes are isolated at day 19 post adoptive transfer and subjected to re-stimulation with HBc₉₃₋₁₀₀ peptides for 6 hours, which is followed by staining of surface markers and intracellular cytokines and flow cytometric analysis of the percentage of the cytokine-secreting CTLs. CD8⁺ CD45.1⁺ cells are gated and defined as transferred CTLs. (D) Bar graph of the percentage of IFN-γ-secreting CTLs (SP) and the percentage of CTLs secreting both IFN-γ and TNF-α (DP). *P<0.05, **P<0.01 and ***P<0.001 (unpaired Student's t-test)

FIGS. 10A-10J depict the Akt signaling facilitates antigen-dependent expansion of CTLs and the antigen clearance in the liver. (A) The percentage of bioluminescence-positive mice equivalent to OVA-positive mice at indicated time points. Kinetics of accumulation of transferred CTLs in the liver (B) and spleen (C) of the mice with intrahepatic expression of the cognate antigen (ovalbumin). 1×10⁵ transduced OT-I CTLs were adoptively transferred into recipient mice receiving hydrodynamic injection (HDI) of a plasmid encoding ovalbumin and luciferase under the control of albumin promoter one day before adoptive transfer. The liver-associated lymphocytes and splenocytes were isolated at day 3, 7 and 14 and subjected to flow cytometric analysis of the percentage and the number of the transferred CTLs. (D) Kinetics of serum ALT in OVA-Luc-positive mice receiving adoptive transfer of 1×10⁵ 2A-CD90.1-engrafted (ctrl) or mAkt1-2A-CD90.1-engrafted (Akt1) OT-I cells. (E, F) Kinetics of accumulation of transferred CTLs in the liver (E) and spleen (F) of the mice as in A. The liver-associated lymphocytes and splenocytes were isolated at day 7, 30 and 63 and subjected to flow cytometric analysis of the percentage and the number of the transferred CTLs. (G) Hematoxylin-and-eosin staining of the liver tissues from E. (H) A representative histogram of BrdU-staining of Akt1-engrafted OT-I CTLs at day 7 and day 63 after adoptive transfer into OVA-Luc-positive recipient mice. (I) Frequency of BrdU⁺ transferred Akt1-engrafted OT-I CTLs at day 7 and day 63 after adoptive transfer into OVA-Luc-positive recipient mice. The recipient mice were given 1 mg BrdU via intraperitoneal injection at day 6 or day 62 after adoptive transfer. The liver-associated lymphocytes and splenocytes were isolated at day 7 and 63 and subjected to flow cytometric analysis of the percentage the BrdU⁺ transferred CTLs. (J) Immunohistochemical analysis of Ki-67 in the liver of OVA-Luc-positive mice receiving adoptive transfer of 2A-CD90.1-engrafted (ctrl) or mAkt1-2A-CD90.1-engrafted (Akt1) OT-I cells. The liver was collected at day 7, day 32 and day 63 after adoptive transfer of 1×10⁵ OT-I CTLs. Scale bars, 40 μm. *P<0.05, **P<0.01 and ***P<0.001 (unpaired Student's t-test), Scale bars, 100 or 40 μm.

FIGS. 11A-11B depict in vivo bioluminescence of mice infected with Ad-Albp-OL. C57BL/6 mice are infected with a recombinant adenovirus carrying genes expressing ovalbumin and luciferase under the control of albumin promoter at different viral doses. The infected mice are monitored for the luciferase expression in the liver by IVIS at indicated time points after infection.

FIGS. 12A-G depict the memory responses of Akt-engineered CD8⁺ T cells. (A) Experimental scheme of re-call response of Akt-engrafted CTLs. (B) The level of serum ALT in the mice receiving adenovirus carrying OVA and luciferase ORFs under the control of albumin promoter (Ad-Albp-OL) and control (ctrl) or Akt1-engrafted OT-I T cells (1×10⁵) at indicated time points post adoptive T cell transfer. (C) The in vivo bioluminescence in mice receiving Ad-Albp-OL, adoptive T cell transfer and hydrodynamic injection (HDI) of a plasmid encoding OVA and luciferase under the control of albumin promoter (pENTRY-Albp-OL) at day 60 after adoptive transfer. (D) Quantification of transferred CTLs in the liver and spleen of the mice receiving Ad-Albp-OL infection and adoptive transfer of ctrl 2A-CD90.1 engrafted OT-I or Akt1-engrafted OT-I followed by HDI of pENTRY-Albp-OL at day 60 after adoptive transfer. The liver-associated lymphocytes and splenocytes were isolated at day 7 after HDI and subjected to flow cytometric analysis of the number of the transferred CTLs. (E) Hematoxylin-and-eosin staining of the liver tissues from D. (F) Immunohistochemical analysis of CD8 in the liver from D. (G) Immunohistochemical analysis of Gr-1 in the liver from D. *P<0.05, **P<0.01 and ***P<0.001 (unpaired Student's t-test), Scale bars, 100 or 40 μm.

FIGS. 13A-13F depict the memory responses of Akt-engineered CD8⁺ T cells. Mice are infected with Ad-Albp-OL, and receive adoptive T cell transfer and HDI of a plasmid encoding OVA and luciferase under the control of albumin promoter (pENTRY-Albp-OL) at day 64 after adoptive transfer. Quantification of (A) transferred CTLs, (B) CD11b⁺NK1.1⁻ myeloid cells, (C) NK1.1⁺ CD3⁻ NK cells and (D) NK1.1⁺ CD3⁺ NKT cells in the liver and spleen of the mice receiving Ad-Albp-OL infection and adoptive transfer of ctrl 2A-CD90.1, Akt1 or Akt2 engrafted OT-I CTLs. The liver-associated leukocytes and splenocytes are isolated at day 7 after adoptive transfer and subjected to flow cytometric analysis of the number of cells. (E) The level of serum ALT in the mice receiving Ad-Albp-OL and ctrl or Akt2-engrafted OT-I T cells (1×10⁵) at indicated time points post adoptive T cell transfer. (F) The in vivo bioluminescence in mice receiving Ad-Albp-OL, adoptive T cell transfer and HDI of a plasmid encoding OVA and luciferase under the control of albumin promoter (pENTRY-Albp-OL) at day 64 after adoptive transfer. *P<0.05, **P<0.01 and ***P<0.001 (unpaired Student's t-test)

FIGS. 14A-14C depict the influence of Akt-engineered CTLs in HCC tumor microenvironment. Immunohistochemical analysis of CD8 (A), F4/80 (B), and cleaved caspase 3 (C) in the liver/tumor of HCC-bearing mice. The HCC development is induced by oncogenes, Akt and N-RasV12 delivered by HDI. At day 31 after HCC induction, the mice are injected with 2×10⁶ Akt2-engrafted OT-I TCR tg CTLs which could recognize an introduced tumor antigen on tumor cells or not (ctrl). The liver/tumor tissues are collected at day 10 after adoptive transfer.

FIGS. 15A-15D depict the anti-tumor capability of Akt-engineered CTLs. The HCC development is induced by oncogenes, Akt and N-RasV12 delivered by HDI. The growth of HCC in mice is monitored by IVIS and the mice with the total flux greater than 10⁹ photons/sec are used as recipients receiving adoptive T cell therapy. The mice are injected with 2×10⁵ ctrl-, Akt1- and Akt2-engrafted HBc₉₃₋₁₀₀-specific CTLs, respectively, which can recognize a surrogate tumor antigen on tumor cells. (A) The in vivo bioluminescence of the mice before and after receiving adoptive T cell transfer. The liver/tumor tissues are collected from mice receiving (B) ctrl-engineered CTLs, (C) Akt1-engineered CTLs or (D) Akt2-engineered CTLs at day 19 after adoptive transfer. *P<0.05, **P<0.01 and ***P<0.001 (unpaired Student's t-test)

FIGS. 16A-16L depict the improved tumor-specific proliferation, cytokine production and cytotoxicity of CAR T cells through overexpression of Akt molecules. (A) Schematic representation of MSCV retroviral constructs used for T-cell engineering contain 5′ and 3′long terminal repeats (LTR), P2A linker peptide sequence (2A) and woodchuck hepatitis virus posttranscriptional regulatory element (WPRE). In pMSCV-mAkt1/Akt2-2A-CAR plasmid, src myristoylation sequence (myr) and mouse AKT1 or AKT2 gene are placed upstream of 2A sequence, followed by chimeric antigen receptor (CAR) ORF e.g. anti-HBs CAR (S-CAR) and anti-CEA CAR In pMSCV-hAkt1/hAkt2-2A-CAR plasmids, the mouse AKT1 or AKT2 gene is replaced by human AKT1 or AKT2 gene. (B) Proliferation of Akt1-engrafted (mAkt1), anti-CEA CAR-engrafted (antiCEA) and Akt1-2A-anti-CEA CAR (mAkt1-antiCEA) CD4⁺ or CD8⁺ T cells. In vitro-activated mouse CD3⁺ T cells transduce with retroviruses carrying mAkt1/mAkt2-2A-CD90.1, anti-CEA CAR or mAkt1/mAkt2-2A-anti-CEA CAR ORF, respectively are co-cultured with LS174T cells. EdU incorporation and detection are applied to monitor the DNA synthesis of the T cells during 22 hours to 28 hours after co-culture. (C, E) IFNγ and (D, F) IL-2 in the supernatant of the co-culture are detected by ELISA. (G, I) Intracellular IFNγ and (H, J) granzyme B staining of the CTLs from the co-culture with LS174T cells for 1 day. (K) Proliferation capability of CTLs in the presence of MDSCs. 2A-CD90.1-engrafted (ctrl) or mAkt1-2A-CD90.1-engrafted OT-I CTLs are re-stimulated with anti-CD3+anti-CD28 beads in the presence of different numbers of MDSCs derived from EL4-tumor-bearing mice. (L) Proliferation capability of CTLs in the presence of MDSCs. 2A-CD90.1-engrafted (ctrl) or mAkt2-2A-CD90.1-engrafted HBc₉₃₋₁₀₀ specific CTLs are re-stimulated with anti-CD3+anti-CD28 beads in the presence of different numbers of MDSCs derived from mouse HCC tumor mass. EdU incorporation and detection are performed to monitor the DNA synthesis of the T cells during 22 hours to 28 hours after co-culture. *P<0.05, **P<0.01 and ***P<0.001 (unpaired Student's t-test)

### DETAILED DESCRIPTION OF THE INVENTION

Unless defined otherwise, all technical and scientific terms used herein have the same meaning as commonly understood by a person skilled in the art to which this invention belongs.

As used herein, the term “OT-I cell” refers to a transgenic line of ovalbumin-specific, CD8⁺ T cell. The transgenic T cell receptor was designed to recognize ovalbumin residues 257-264 in the context of H-2K^{b} and used to study the role of peptides in positive selection and the response of CD8⁺ T cells to antigen.

As used herein, the term “AdHBV” refers to the adenovirus carrying HBV genome. HBV-infected mouse model can be established by hydrodynamic injection (HDI) of the HBV genome into the tail vein.

As used herein, the term “HBcAg” refers to a hepatitis B viral protein, which is an antigen that can be found on the surface of the nucleocapsid core of the hepatitis B virus.

As used herein, the term “HBeAg” refers to a hepatitis B viral protein, which is an antigen that can be detected in the serum of mice with HBV infection established by AdHBV infection or HDI of a plasmid harboring the HBV genome.

The DNA or RNA molecules in this present invention can be amplified through plasmid amplification, in vitro transcription or in vitro synthesis and transfected into target cells through electroporation, liposome or other chemical vehicles.

The aforementioned target cells for genetic modification can be T cells, nature killer cells, hematopoietic stem cells, embryonic stem cells and pluripotent stem cells from various species. These cells can be modified by viral transduction or DNA (or RNA) transfection.

The recombinant viral or transposon vectors can be retroviruses, lentiviruses, adenoviruses, adeno-associated viruses, other related viruses and various transposon systems can be used in transduction or integration of transgenes.

To investigate the mechanism of how liver microenvironment can influence secondary expansion of virus-specific CTL population in the liver, in vitro-activated HBV specific CD8⁺ T cells are adoptively transferred into HBV carrier mice and the change of the serum level of HBV antigen in these mice is detected. It is found that most of the mice failed to eliminate persistent HBV infection within 42 days. The cell number and expression level of exhaustion markers including PD-1, TIM-3, and LAG-3 on the adoptively transferred CTLs in the liver and in the spleen of the HBV carrier mice are further detected. The cell number of adoptively transferred HBV-specific CTLs increases in the liver but not in the spleen. The HBV-specific CTLs in both the liver and the spleen express higher levels of PD-1 and LAG-3 than endogenous CD8⁺ T cells; however, the splenic HBV-specific CTLs express lower levels of PD-1. TIM-3 and LAG-3 than intrahepatic compartments. Those results demonstrate that the exposure to HBV antigens expressed in the liver microenvironment induces T-cell exhaustion of HBV-specific CTLs.

The immune checkpoints PD-1 and CTLA-4 are shown to prevent Akt phosphorylation % activation during TCR triggering through recruitment of SHP-1/2 and activation of PP2A, respectively. We therefore examine whether Akt signaling is critical to intrahepatic expansion and differentiation of CD8⁺ T cells. Mouse AKT1, AKT2 and AKT3 genes are cloned, respectively, with addition of src myristoylation sequence in the upstream of AKT genes to ensure the membrane targeting and being constitutively active of the Akt molecules. The expression of exogenous myristoylated Akt isoforms are detected by Western blot in Akt-engineered CTLs but not in the control T cells. CTLs are engrafted with three different kinds of Akt, respectively, all show Akt phosphorylation at Ser473 and only those are engrafted with Akt1 or Akt2 show Akt phosphorylation at Thr308.

To examine whether overexpression of Akt is related to intrahepatic survival or secondary expansion of CTLs in response to antigen stimulation, the ovalbumin (OVA) and luciferase expression are induced in the liver of recipient mice by hydrodynamic injection (HDI) of a plasmid encoding OVA and luciferase. After being adoptive transfer into the recipient mice, Akt1- and Akt2-engineered CTL populations expand vigorously in the liver and the spleen. There is more than 250,000-fold for Akt1 CTLs and 950,000-fold for Akt2-CTLs cell numbers found in the liver in comparison with that of ctrl-CTLs at day 7 after adoptive transfer.

Owing to the huge contribution of immune checkpoints on T-cell exhaustion in the liver during chronic viral infection, the inventors therefore examine whether Akt signaling have an influence the expression of immune checkpoint molecules on HBV-specific CTLs per se. After in-vitro activation and transduction, the Akt- or ctrl-engineered HBc₉₃₋₁₀₀-specific CTLs are adoptively transferred into AdHBV-infected mice and analyzed the surface expression of immune checkpoint molecules on the CTLs at day 6 and day 19 after adoptive transfer. Hepatic ctrl-CTLs expressed high level of PD-1, TIM-3 and LAG-3 at day 19 after adoptive transfer, whereas Akt1-CTLs and Akt2-CTLs expressed significantly less PD-1, TIM-3 and LAG-3 at day 19 post adoptive transfer.

To further investigate whether these Akt-CTLs can overcome the suppressive mechanisms in the liver and mediate clearance of persistent HBV infection, the ctrl- or Akt1-engineered HBc₉₃₋₁₀₀-specific CTLs are adoptively transferred into HBV carrier mice. Akt1-CTLs but not ctrl-CTLs eliminate persistent HBV infection within 14 days after being adoptive transferred into HBV carrier mice. The Akt1-CTLs are mainly in the liver rather than in the spleen and disperse to the spleen after antigen clearance. There are less HBcAg-positive hepatocytes but more cleaved caspase 3-positive apoptotic hepatocytes detected in the liver of mice receiving Akt1-CTLs than in the liver of mice receiving ctrl-CTLs. After clearance of antigen, the mononuclear cells reduce and HBcAg-positive hepatocytes as well as cleaved caspase 3-positive hepatocytes are no longer detected in the liver of mice receiving Akt1-CTLs. The ctrl-CTLs fail to clear HBV and do not induce significant inflammation after being adoptively transferred into HBV carrier mice. Akt2-CTLs expand vigorously when encountering the cognate antigen in vivo, and prevent T-cell from exhaustion. Also, Akt2-CTLs exhibit strong cytotoxic function and are more efficient to clear HBV infection than ctrl CTLs.

The capability of Akt-engineered CTLs in killing of hepatocellular carcinoma (HCC) is further examined. The tumor antigen-specific Akt2-engrafied CD8⁺ CTLs can accumulate in the tumor sites as well as in the liver at day 10 after adoptive transfer into HCC-bearing mice. These Akt2-CTLs change the tumor microenvironment and to attract or activate the surrounding F4/80⁺ macrophages in tumor sites. Furthermore, a lot of cleaved caspase 3-positive tumor cells are detected in the mice receiving Akt2-CTLs but not in ctrl mice. Elevated serum ALT in the mice with Akt2-CTLs is also observed but not in ctrl mice (118.1 U/L vs. 22.8 U/L). It can be concluded that Akt2 activation enables CTLs to have strong effector functions and be able to kill tumor cells in the liver. This is probably through CTLs' own cytotoxic capability or through release of cytokines to activate the anti-tumor functions of tumor-associated macrophages.

To further explore the potential application of Akt molecules on cancer immunotherapy, the plasmids carrying human or mouse Akt1 or Akt2 genes and anti-CEA (Carcinoembryonic antigen) chimeric antigen receptor (CAR) are constructed. CEA are glycosyl phosphatidyl inositol (GPI) cell-surface-anchored glycoproteins and are critical to the dissemination of colon carcinoma cells. The modified CTLs are co-cultured with a colorectal adenocarcinoma cell line, LS174T. Both CD4⁺ and CD8⁺ T cells with the engraftment of anti-CEA CAR can respond to stimulation of LS174T and proliferate. Additional active Akt1 expression in anti-CEA CAR engrafted T cells can promote the proliferation capability of both CD4⁺ and CD8⁺ T cells. More IL-2 and IFNγ are detected in the culture medium of co-culture of LS174T cell line with T cells expressing anti-CEA CAR and Akt1 or Akt2 molecules compared to that of LS174T and T cells expressing solely anti-CEA CAR. Intracellular staining of IFNγ and granzyme B of the CD8⁺ T cells co-culture with LS174T cells also proves that Akt1 or Akt2 overexpression can enhance the cytokine production and cytotoxicity in CTLs. Strikingly, Akt1- and Akt2-overexpressing CTLs, respectively are shown to have the capability to overcome the proliferative arrest induced by myeloid-derived suppressor cells (MDSCs), which strongly suggests that the potential application of Akt molecules on T-cell engineering technology e.g. CAR T cells for immunotherapy.

The following examples are offered by way of illustration and not by way of limitation. The mAkt isoforms are utilized in the mouse model as a demonstration in this present invention, but is not intended to limit the scope of the invention.

### Example 1: Cytotoxic T Lymphocytes Undergo Exhaustion in the Liver

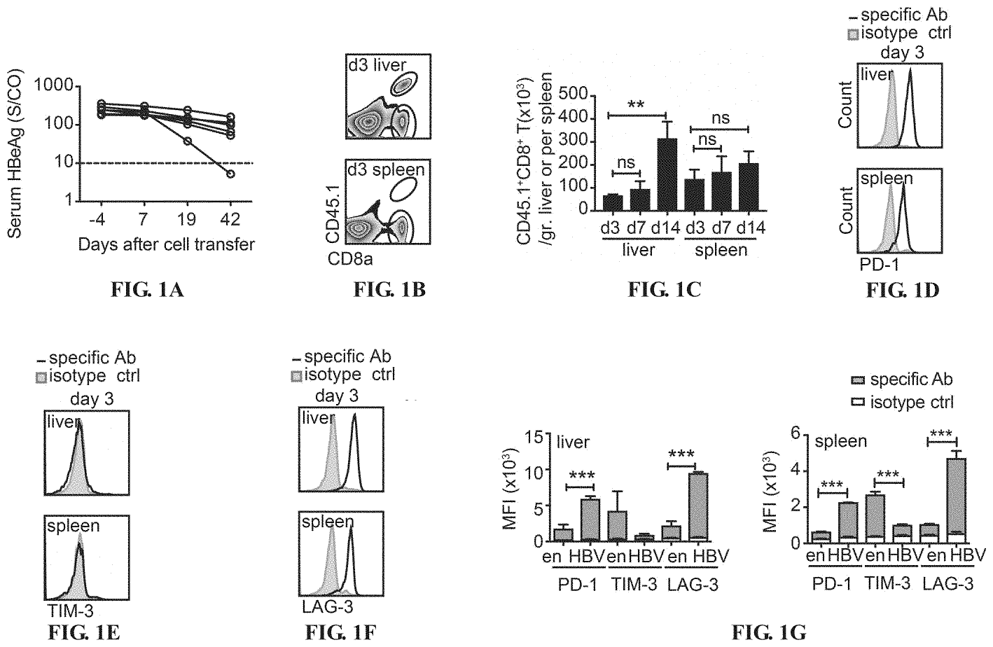

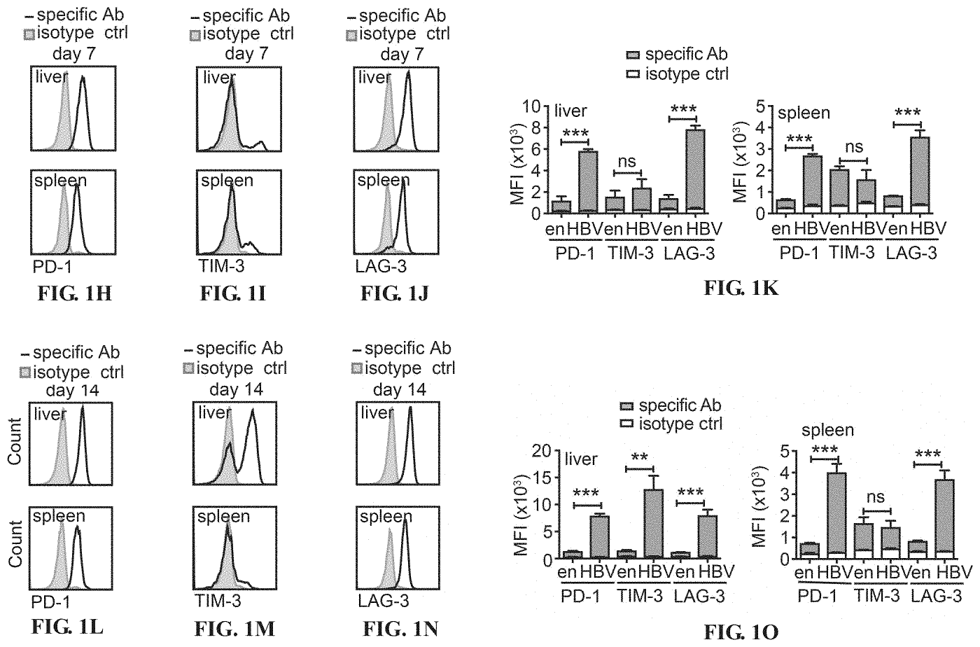

In vitro-activated CD45.1 HBc₉₃₋₁₀₀ specific CD8⁺ T cells are adoptively transferred into congenic C57BL/6 mice infected with the adenovirus carrying HBV genome (AdHBV), and the change of the serum level of HBeAg in these mice is detected. It is found that most of the mice failed to eliminate persistent HBV infection within 42 days (FIG. 1A).

The cell number and expression level of exhaustion markers are further detected, which including PD-1, TIM-3, and LAG-3 on the adoptively transferred CTLs in the liver and in the spleen of the HBV carrier mice at day 3, day 7 and day 14 post adoptive transfer. The cell number of adoptively transferred HBV-specific CTLs increases from day 3 to day 14 in the liver but not in the spleen (FIGS. 1B and 1C).

Endogenous CD8⁺ T cells are used as a reference population for evaluation of the expression level of these exhaustion markers on HBV-specific CTLs. The HBV-specific CTLs in both the liver and the spleen express higher levels of PD-1 and LAG-3 than endogenous CD8⁺ T cells but no or little TIM-3 at day 3 and day 7 post adoptive transfer (FIGS. 1D-1K).

The splenic HBV-specific CTLs express lower levels of PD-1 and LAG-3 than intrahepatic compartments at all time points (FIGS. 1D-1O). The HBV-specific CTLs gradually express TIM-3 after adoptive transfer and reach to a higher level of expression than endogenous CD8⁺ T cells at day 14 in the liver but not the spleen (FIGS. 1E, 1G, 1I, 1K, 1M and 1I).

### Example 2: Expression of Constitutively Active Akt Isoforms in CTLs

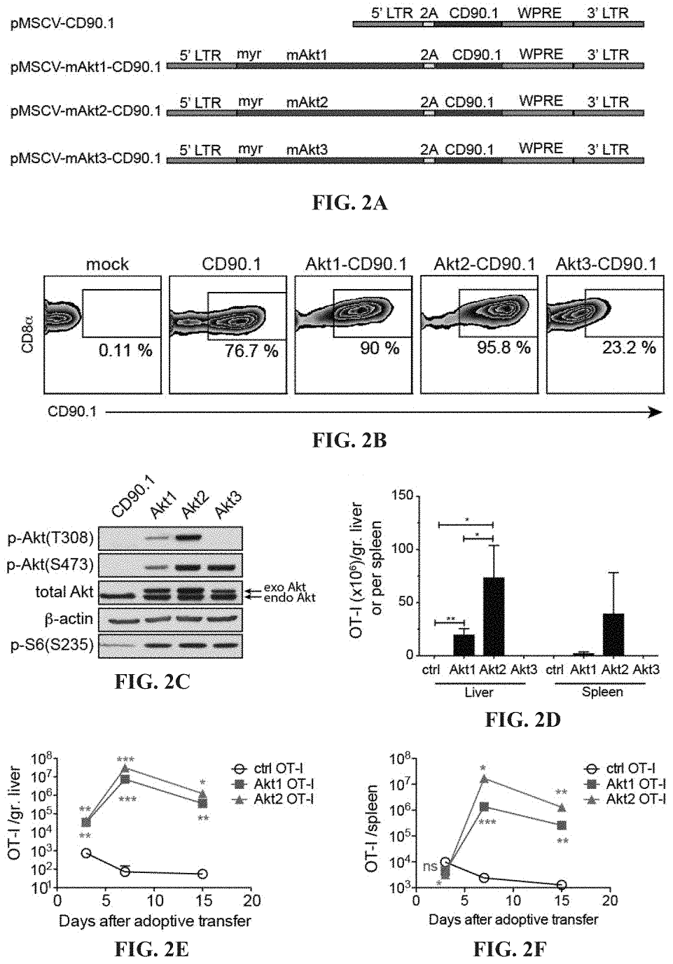

Murine stem cell retroviral (MSCV) system is chosen for delivery of genes into T lymphocytes due to its high efficiency to transduce hematopoietic cell lineages. A pMSCV-CD90.1 plasmid is generated from a replacement of hygromycin resistance gene by p2A peptide sequence and mouse CD90.1 open reading frame (ORF) with the woodchuck hepatitis virus posttranscriptional regulatory element (WPRE) in the 3′ untranslated region of CD90.1 gene to enhance the expression of the transgenes. The CD90.1 gene and WPRE sequence are amplified from pLKO_TRC024 plasmid (RNAi core lab, Taipei, Taiwan). Mouse AKT1 (SEQ ID NO: 1). AKT2 (SEQ ID NO: 3) and AKT3 (SEQ ID NO: 5) genes are cloned, respectively, through PCR using cDNA from mouse 4T1 breast cancer cells with addition of src myristoylation sequence by PCR primer in the upstream of AKT genes to ensure the membrane targeting and being constitutively active of the Akt molecules. The myristoylation sequence and AKT genes are linked, respectively, to mouse CD90.1 gene by p2A peptide sequence in pMSCV-CD90.1 to result in pMSCV-mAkt1-CD90.1, pMSCV-mAkt2-CD90.1 and pMSCV-mAkt3-CD90.1. The expression cassette is flanked by 5′ and 3′ MSCV long terminal repeats (LTRs). The 4 plasmids are used to produce recombinant retroviruses carrying mouse AKT1. AKT2, AKT3 or control CD90.1 gene, respectively (FIG. 2A).

Splenic ovalbumin-specific TCR tg OT-I CD8⁺ T cells are activated by anti-CD3+anti-CD28 beads, subsequently transduced by recombinant retroviruses and are subjected to surface marker staining using antibody recognizing CD90.1 as a tag for transgene expression followed by flow cytometric analysis. Around 75% to 95% of the effector CD8⁺ T cells are transduced with retroviruses carrying CD90.1, AKT1-CD90.1 or AKT2-CD90.1 gene, positive for CD90.1, whereas only 23% of the cells are transduced with retroviruses carrying AKT3-CD90.1 gene expressed low level of CD90.1 (FIG. 2B).

It has been shown that the expression patterns of the three Akt isoforms are different. Akt1 (SEQ ID NO: 1) and Akt2 (SEQ ID NO: 3) are ubiquitously expressed in nearly all tissues whereas Akt3 (SEQ ID NO: 5) are mainly expressed in brain and testes. The tissue specific expression manner of Akt isoforms may explain the low expression of Akt3 by the CD8⁺ T cells. The expression of exogenous myristoylated Akt isoforms is detected by Western blot in Akt-engineered CTLs but not in the control T cells. CTLs engrafted with three different kinds of Akt, respectively, all show Akt phosphorylation at Ser473 and only those which are engrafted with Akt1 or Akt2 show Akt phosphorylation at Thr308 (FIG. 2C).

### Example 3: Akt Signaling Facilitates Antigen-Dependent Expansion of CTLs in the Liver

Ovalbumin (OVA) and luciferase expression are induced in the liver of recipient mice by hydrodynamic injection (HDI) of a plasmid encoding OVA and luciferase under the control of albumin promoter (pENTRY-Albp-OL). After being adoptive transfer into the recipient mice, Akt1- and Akt2- but not Akt3-engineered CTL or CD90.1-engineered (ctrl) populations expanded vigorously in the liver and the spleen.

These Akt1- or Akt2-CTLs underwent vigorous proliferation and yielded 23 million (Akt1) and 113 million (Akt2) splenic and intrahepatic CTLs in total, respectively, after antigen stimulation in the liver (FIG. 2D) despite that there only 0.1 million activated CD8⁺ T cells are originally injected into the recipient mice. Most of the ctrl CTLs disappear after adoptive transfer probably due to the lack of co-stimulation, growth signals or the suppressive liver microenvironment.

The massive expansion of Akt1- or Akt-2-OT-I CTLs is further confirmed in a time kinetic experiment (FIGS. 2E and 2F). Akt2-CTLs are found to be more potent in expansion in the liver and in the spleen than ctrl- or Akt1-CTLs (FIGS. 2D-F). Moreover, Akt1-CTLs preferentially locate in the liver rather than the spleen (FIGS. 2D-F).

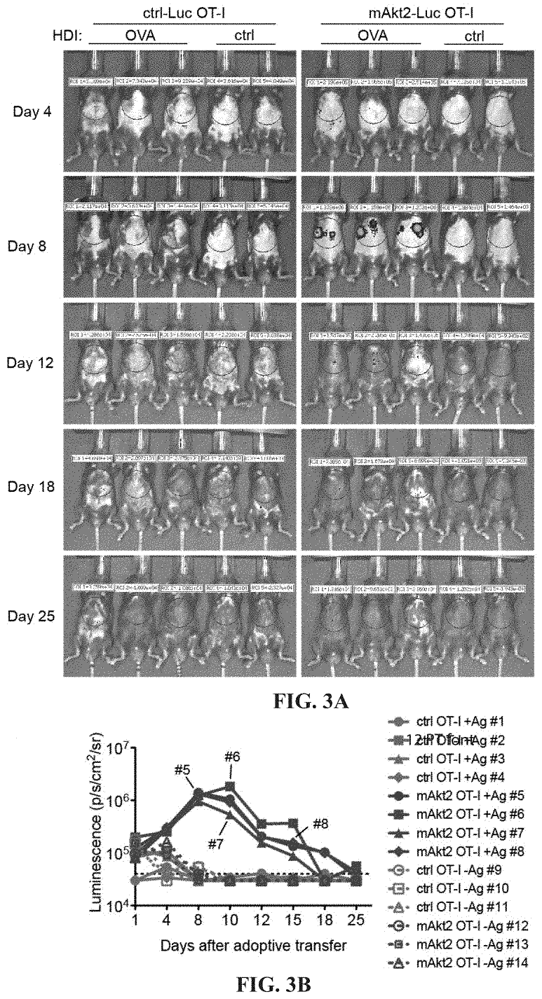

Therefore, Akt constructs with co-expression of luciferase instead of CD90.1 are designed for monitoring the distribution and expansion of Akt-engineered CTLs. Control (ctrl) Luc-CTLs and Akt2-Luc-CTLs are delivered respectively, to mice with or without OVA expression in their livers and only observed TCR signaling-dependent Akt2-Luc-CTL accumulation in the liver but not in other organs or in mice without antigen expression in the liver (FIGS. 3A-3B), which suggests that signaling through constitutively active Akt can assist massive CTL expansion only in combination with TCR triggering and these Akt-CTLs undergo T-cell contraction after the clearance of antigen. Again, the ctrl CTLs fail to expand in respond to antigen stimulation in the liver (FIGS. 3A-3B).

### Example 4: Akt Signaling Suppresses the Expression of Immune Checkpoint Molecule on CTLs

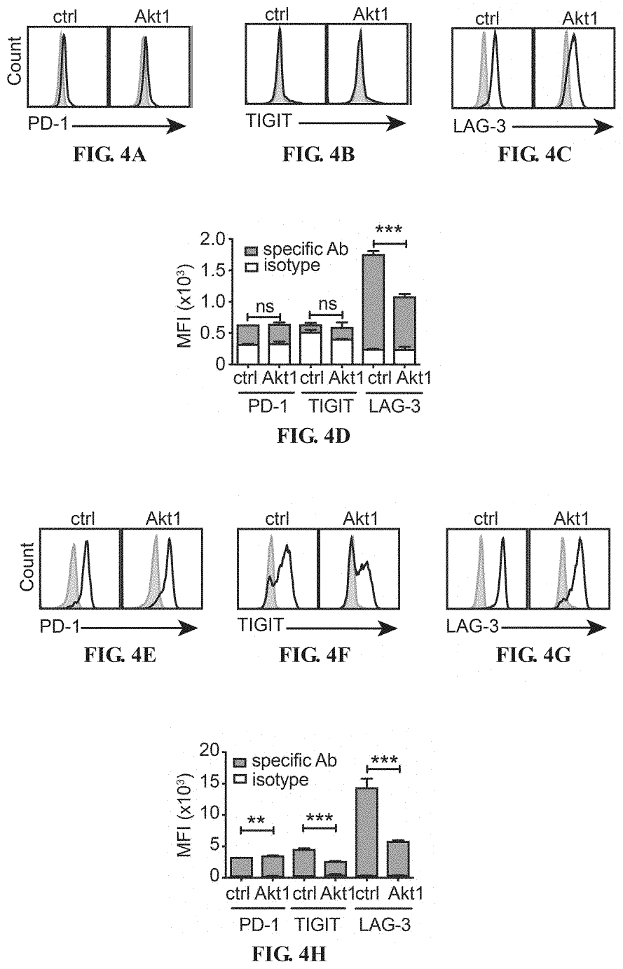

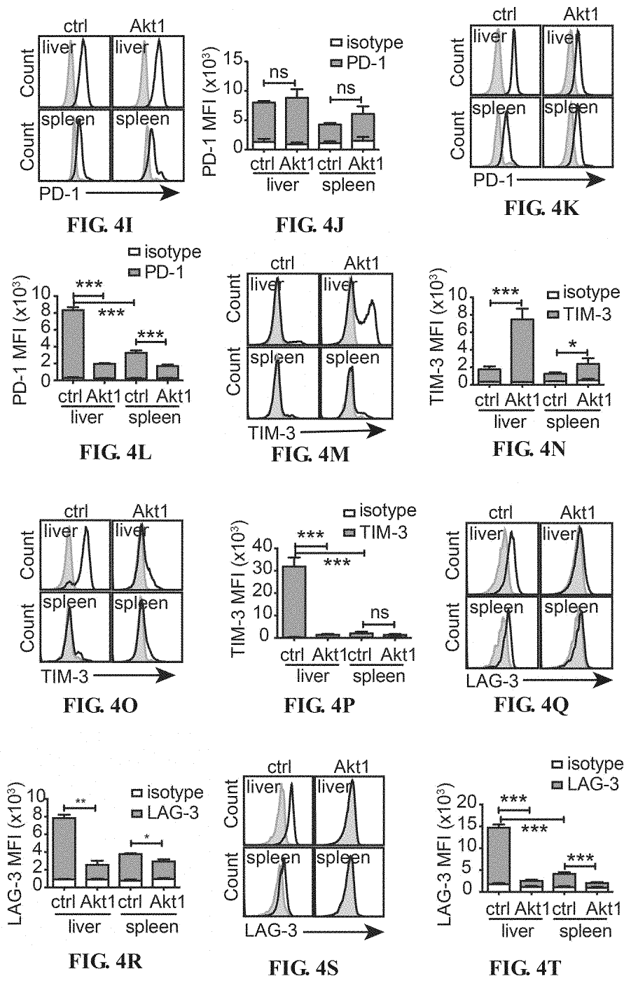

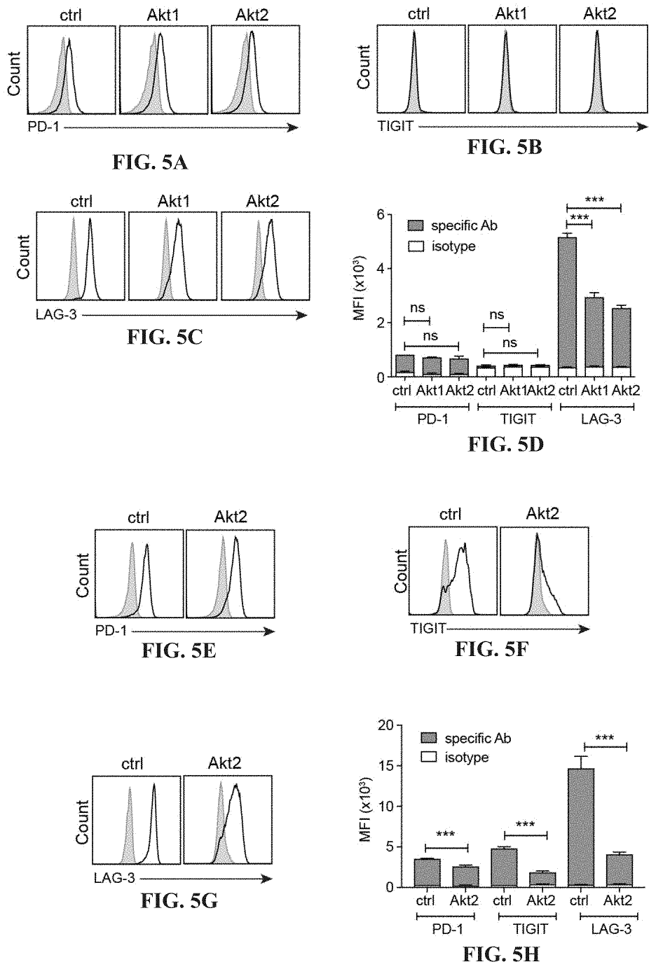

After in-vitro activation and transduction, HBc₉₃₋₁₀₀-specific CD8⁺ T cells at day 3 after activation are analyzed for their surface expression of various immune checkpoints. The overexpression of constitutively active Akt12 does not change the surface expression of PD-1 and TIGIT (FIGS. 4A, 4B and 4D, FIGS. SA, SB and SD): however, it significantly reduces the expression of LAG-3 on the surface of Akt1- and Akt2-CTLs (FIGS. 4C and 4D, FIGS. 5C and 5D).

These CTLs at day 3 after anti-CD3/anti-CD28 bead activation may have returned to resting status with low or no expression of immune checkpoints e.g. PD-1 and TIGIT except LAG-3. Therefore, the expression level of these immune checkpoints on CTLs after re-stimulation is measured. Expression of PD-1 is rapidly detected on ctrl-, Akt1- and Akt2-CTLs (FIGS. 4E and 4H, FIGS. SE and 5H) and slightly higher on Akt1-CTLs than ctrl-CTLs (FIGS. 4E and 4H). However, the expression of PD-1 on Akt2-CTLs is lower than ctrl-CTLs (FIGS. SE and 5H). Notably, the Akt1- or Akt2-CTLs maintain relatively lower expression of LAG-3 and TIGIT than ctrl-CTLs after re-stimulation with anti-CD3/CD28 beads for 24 hours (FIGS. 4F-H, FIGS. 5F-H).

To further investigate whether the regulation of immune checkpoints on CTLs by Akt signaling also happens in liver microenvironment, the Akt1- or ctrl-engineered HBc₉₃₋₁₀₀-specific CTLs are adoptively transferred into AdHBV-infected mice and analyzed the surface expression of immune checkpoint molecules on the CTLs at day 6 and day 19 after adoptive transfer. The expression patterns of each examined immune checkpoints are quite different. Both intrahepatic Akt1- and ctrl-engineered CTLs at day 6 after adoptive transfer express high level of PD-1 when encountering the cognate antigen in the liver, but the PD-1 expression is down regulated in the Akt1-CTLs at day 19 after adoptive transfer (FIGS. 4I-L).

At day 6 after exposure to HBV, a certain proportion of the hepatic Akt1-CTLs expressed high level of TIM-3, whereas splenic CTLs and ctrl-CTLs in liver express lower level of TIM-3 at this time point, which suggests a stronger TCR triggering in Akt1-CTLs than in ctrl-CTLs (FIGS. 4M and 4N). However, during day 6 to day 19, the expression of TIM-3 decreases in hepatic Akt1-CTLs, whereas it increases dramatically in the ctrl-CTLs in liver but not in the CTLs in spleen (FIGS. 4M-P).

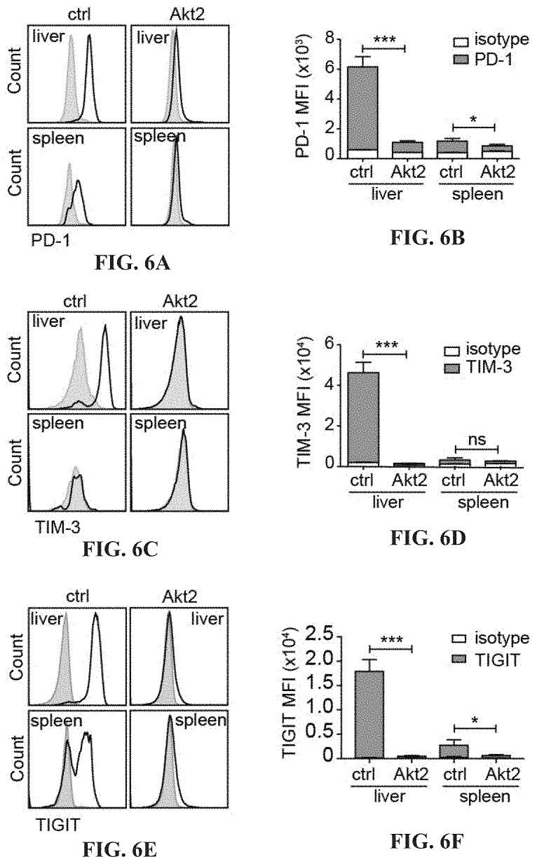

Hepatic ctrl-CTLs express high level of LAG-3 at both day 6 and day 19 after adoptive transfer, whereas Akt1-CTLs express less LAG-3 on their surface during the whole period (FIGS. 4R-T). Akt2-CTLs also show dramatic down-regulation of PD-1, TIM-3 and TIGIT (FIGS. 6A-6F).

These in-vitro and in-vive data clearly demonstrate that Akt signaling possesses very few influence on PD-1 expression but positively regulates TIM-3 expression on CTLs during early TCR signaling. We further prove that augmentation of Akt signaling prevents the expression of LAG-3 and TIGIT on CTLs in the liver during persistent HBV infection, which may contribute the robust expansion and potent effector functions of Akt-CTLs against HBV.

The higher expression of PD-1 and TIM-3 on Akt-CTLs than on ctrl-CTLs after re-stimulation in vitro and in vivo strongly suggests a stronger TCR triggering in Akt-CTLs than that in ctrl-CTLs and also excludes the lack of antigen stimulation at this early time point, which results in down-regulation of LAG-3 and TIGIT. The early expression of TIM-3 on Akt-CTLs may additionally involve in the augmentation of effector functions of Akt-CTLs to combat HBV infection. The reduced expression of immune checkpoints on Akt-engineered CTLs at the later time point may result from the lack of antigen stimulation due to the intense effector functions of Akt-CTLs, which facilitates the early removal of the HBV antigen from the liver.

### Example 5: Akt Signaling in CTLs Enhances their Effector Functions and Facilitated HBV Clearance

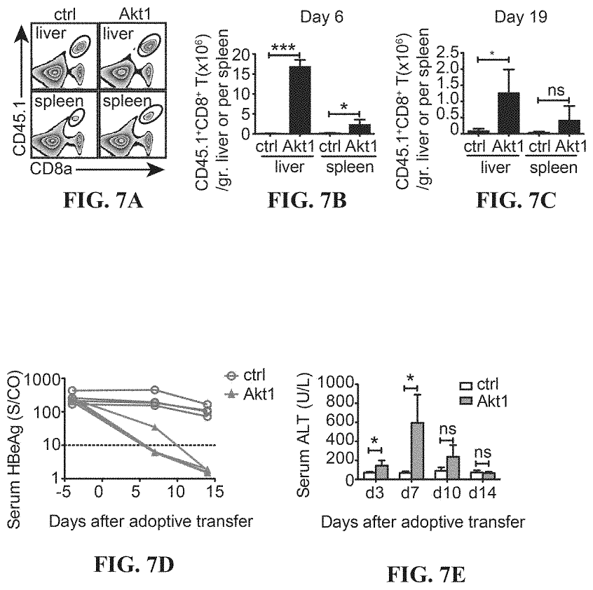

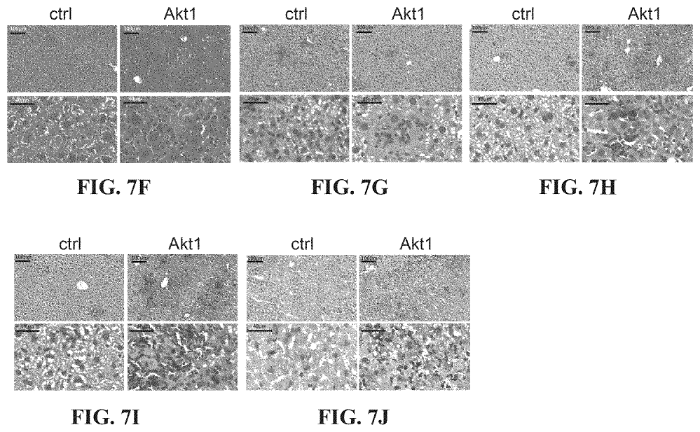

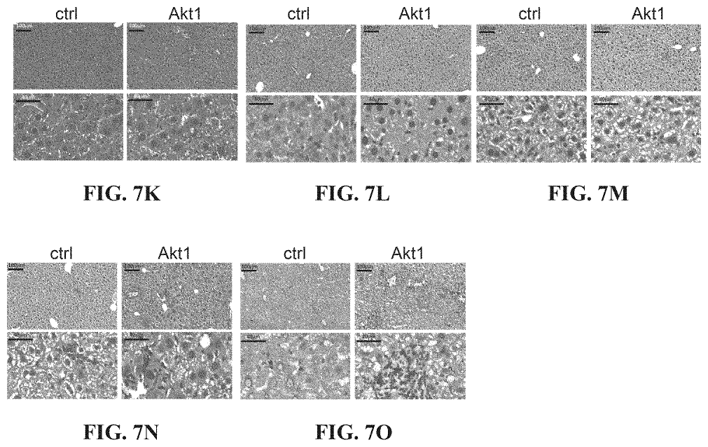

The cell number of adoptively transferred ctrl- or Akt1-engineered HBc₉₃₋₁₀₀-specific CTLs in the liver and in the spleen of HBV carrier mice is measured, and there are more Akt1-CTLs than ctrl-CTLs recovered from the liver at both of day 6 and day 19 after adoptive transfer (FIGS. 7A-C).

Akt1-CTLs but not ctrl-CTLs eliminate persistent HBV infection within 14 days after being adoptive transferred into HBV carrier mice (FIG. 7D). These Akt1-CTLs have better cytotoxic functions than ctrl-CTLs, which is revealed by the elevated serum ALT level from day 3 to day 7 (FIG. 7E). The Akt1-CTLs are mainly in the liver rather than the spleen at day 6 post adoptive transfer and dispersed to the spleen after antigen clearance (FIGS. 7B and 7C). From the H&E staining of the liver sections, a huge number of mononuclear cells in the liver sinusoid of mice receiving Akt1-CTLs at day 6 are observed after adoptive transfer (FIG. 7F).

Immunohistochemical staining is performed to visualize the HBcAg or cleaved caspase 3 expression by hepatocytes and immune cells in the liver of HBV carrier mice. There are less HBcAg-positive hepatocytes but more cleaved caspase 3-positive apoptotic hepatocytes detected in the liver of mice receiving Akt1-CTLs than in the liver of mice receiving ctrl-CTLs at day 6 after adoptive transfer (FIGS. 7G and 7H). The apoptotic hepatocytes or HBcAg⁺ hepatocytes are surrounded by mononuclear cells in the liver of mice receiving Akt1-CTLs which suggests a cytotoxic role of these Akt1-CTLs against HBV-infected hepatocytes (FIGS. 7G and 7H). There are more Gr-1⁺ myeloid cells and adoptively transferred CTLs (CD45.1⁺) detected in the liver of mice receiving Akt1-CTLs than in the liver of mice receiving ctrl-CTLs at day 6 (FIGS. 7I and 7J).

After clearance of antigen, the liver histology appears back to normal, the mononuclear cells reduce and HBcAg-positive hepatocytes as well as cleaved caspase 3-positive hepatocytes are no longer detected in the liver of mice receiving Akt1-CTLs (FIGS. 7K-M). The number of Gr-1⁺ myeloid cells also reduces whereas a significant number of CD45.1⁺ adoptively transferred CTLs still exists in the liver of mice receiving Akt1-CTLs (FIGS. 7N and 7O). The ctrl-CTLs fail to clear HBV (FIGS. 7D, 7G and 7L) and cannot induce significant inflammation after being adoptively transferred into HBV carrier mice (FIGS. 7E-7O).

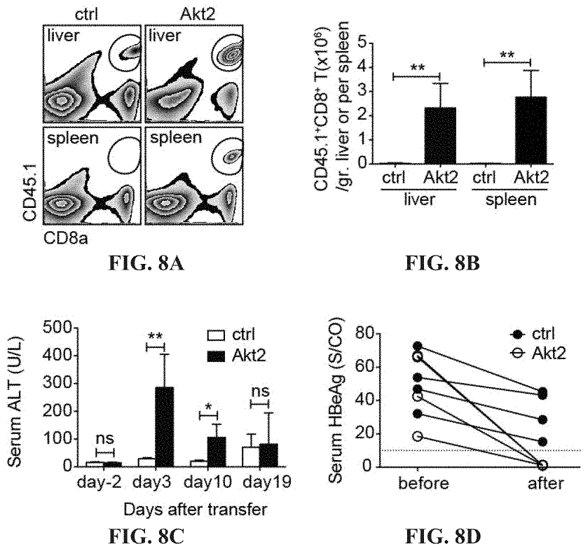

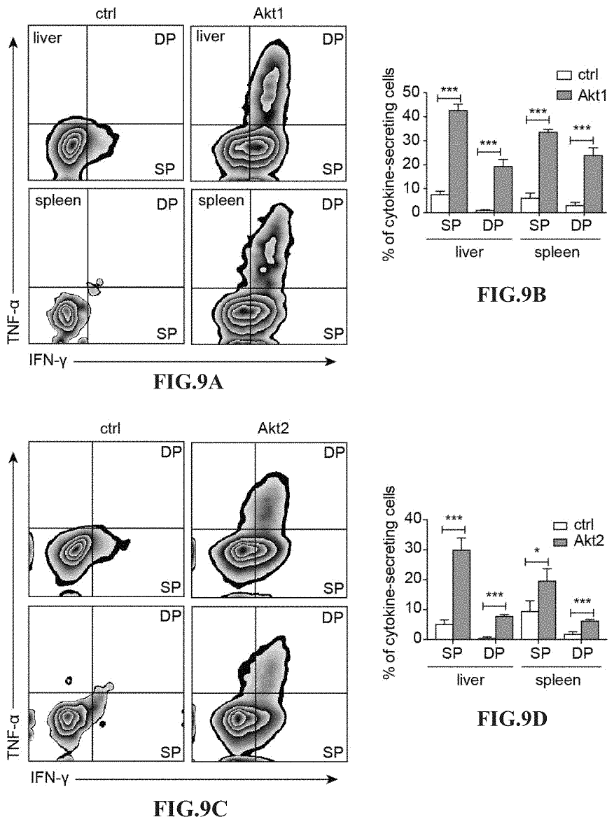

Akt2-CTLs also expand vigorously when encountering the cognate antigen in vive (FIGS. 8A and 8B), prevent T-cell exhaustion (FIGS. 6A-6F), exhibited strong cytotoxic function (FIG. 5C) and are more efficient to clear HBV infection than ctrl CTLs (FIG. 8D). Akt1- and Akt2-CTLs are found more capable to produce IFN-γ and TNF-α than ctrl-CTLs after ex vivo re-stimulation with the specific HBc peptide (FIGS. 9A-D), which is consistent with their capability to induce inflammatory responses as seen in FIGS. 7A-7O.

### Example 6: Akt1 Drives Only TCR Signaling-Dependent Expansion and Facilitates the Self-Renewal of CTLs

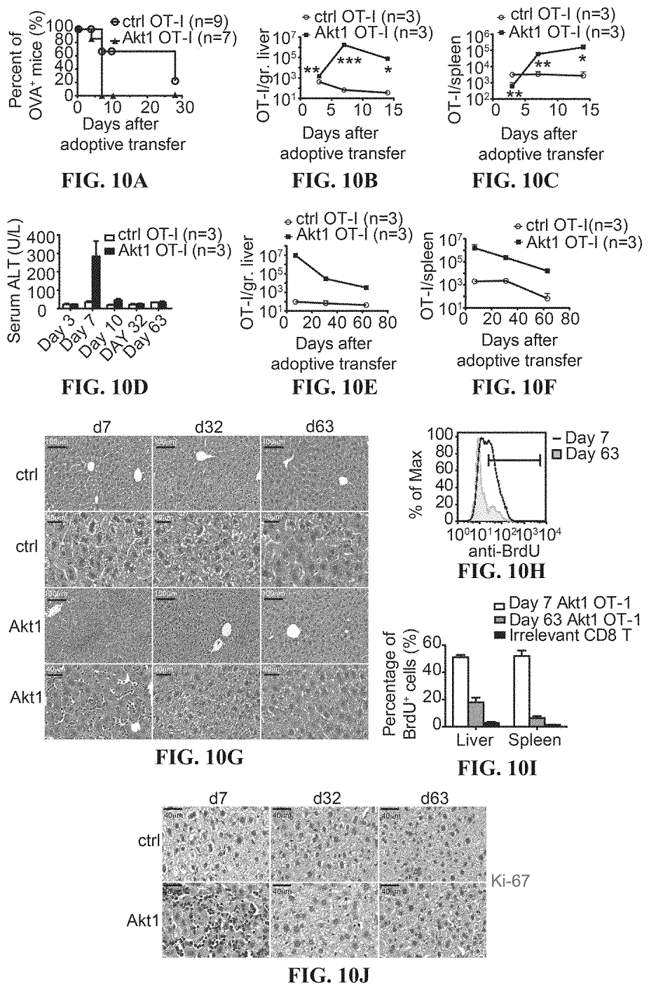

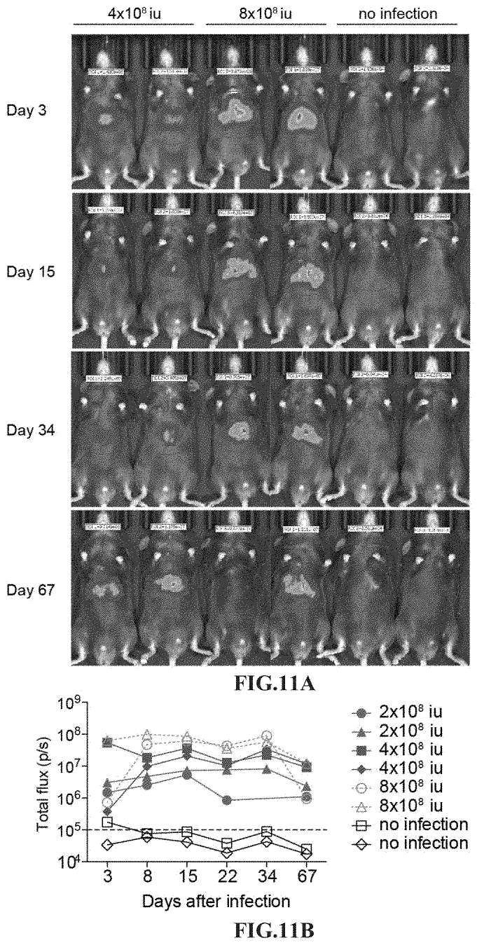

We further examined the capability of the engineered CTLs to eliminate antigen from the liver through the measurement of the bioluminescence in the liver of the recipient mice. The loss of bioluminescence represented the clearance of antigen from the liver. We found that Akt1-OT-I CTLs were more efficient than ctrl OT-I CTLs to eliminate OVA from the liver (FIG. 10A). They cleared the antigen within 7 days, which was also the peak of the expansion of the cell population in the liver (FIGS. 10B and 10C). These Akt1-OT-I CTLs were more capable to execute cytotoxicity toward OVA-expressing hepatocytes than ctrl CTLs did, which was revealed by the elevated serum ALT level of mice receiving Akt1-CTLs at day 7 post adoptive transfer (FIG. 10D).

Being concerned about that the overexpression of Akt molecules in CTLs may potentially induced oncogenic property of the transduced cells, we therefore monitored the numbers of intrahepatic and splenic transferred CTLs and serum ALT levels in the mice receiving ctrl-CTLs and Akt1-CTLs for a longer period of time. The serum ALT levels of mice receiving Akt1-CTLs decreased to normal levels after the clearance of antigens and cell numbers of Akt1-CTL also dropped at least 5000-fold from day 7 to day 63 (FIGS. 10D-F). We detected a lot of mononuclear cells lying in the liver sinusoid of mice receiving Akt1-CTLs but not ctrl-CTLs at day 7 post adoptive transfer (FIG. 10G). The architecture of the livers of mice receiving Akt1-CTLs returned to normal at day 32 and day 63 after clearance of antigen (FIG. 10G).

We further analyzed the proliferation capability of these adoptively transferred Akt1-CTLs or endogenous CD8⁺ T cells at day 7 and day 63, respectively and found that even in the absence of antigen, the Akt1-CTLs could still undergo higher grade DNA synthesis to sustain self-renewal than endogenous CD8⁺ T cells did, which explained the maintenance of the cell number after clearance of antigen (FIGS. 10H and 10I). These Akt1-CTLs in the liver sinusoid were all Ki-67-positive at day 7 after adoptive transfer, which demonstrated that they were undergoing vigorous proliferation and were barely detected in the liver sinusoid at day 32 and day 63 after adoptive transfer (FIG. 10J).

### Example 7: Akt Signaling Facilitates Development of T Cell Memory

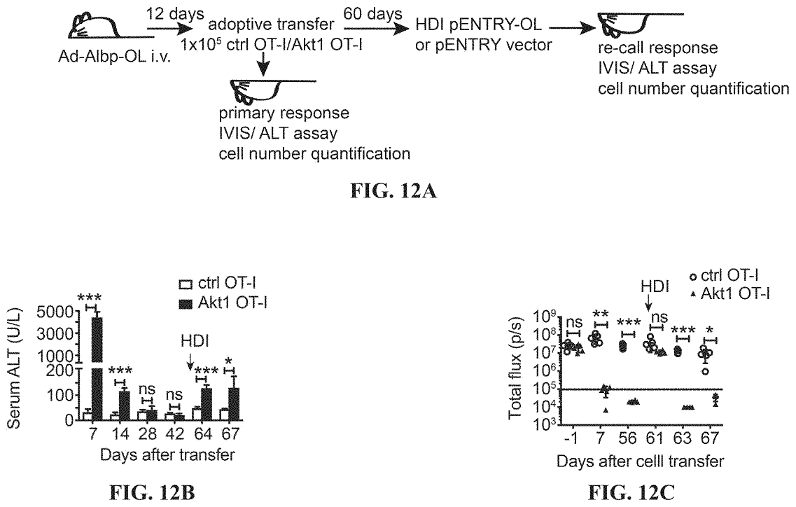

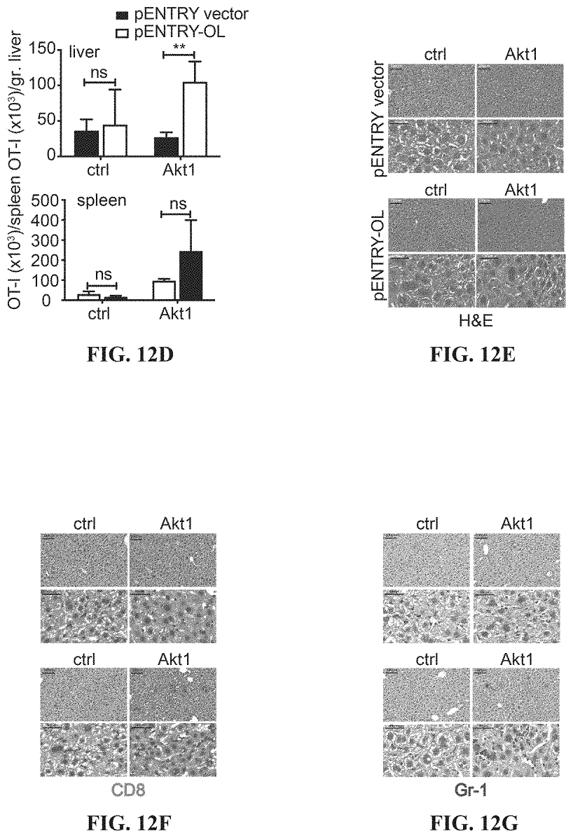

It has been shown that virus-infected hepatocytes were highly sensitive to CTL-induced cytotoxicity. The liver microenvironment after HDI may not completely mimic that during viral infection. We therefore established an adenovirus (Ad-Albp-OL)-based liver infection mouse model with persistent expression of OVA and luciferase only in the liver under the transcriptional control of albumin promoter in order to study the functions of Akt in CTLs under the circumstance of intrahepatic persistent viral infection. We first titrated the viral doses for infection and found that infection with 2×10⁸ and 4×10⁸ iu of Ad-Albp-OL, respectively, could induce stable expression of luciferase for more than 2 months (FIGS. 11A-11B). We then infected mice with 4×10⁸ iu of Ad-Albp-OL, adoptively transferred Akt- and ctrl-CTLs, respectively, into the mice and performed several analyses following the experimental scheme showed in FIG. 12A.

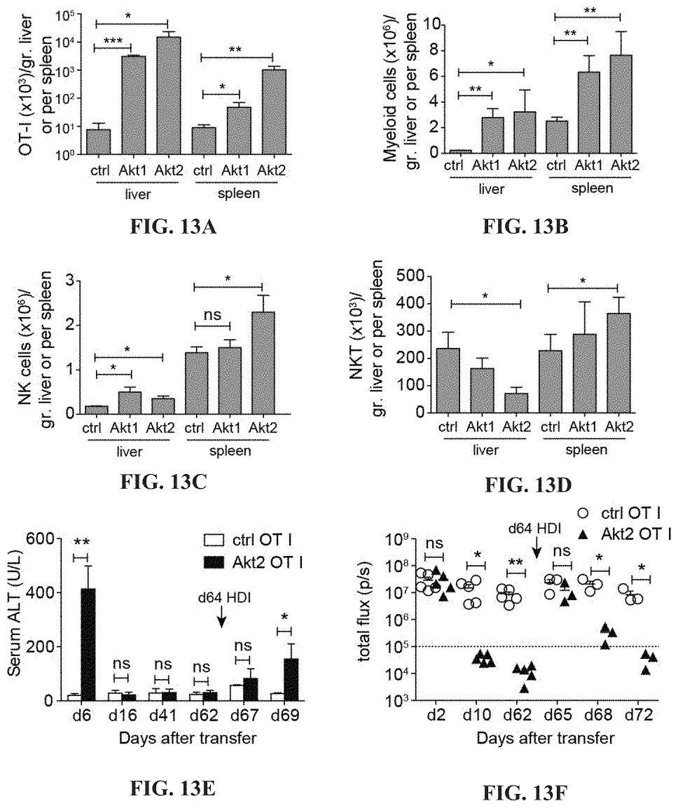

Similar to the data from HDI model, there were more Akt1- or Akt2-CTLs than ctrl-CTLs detected in the liver and in the spleen of Ad-Albp-OL-infected mice at day 7 after adoptive transfer (FIG. 13A). The inflammation induced by Akt1- or Akt2-CTLs further promoted the innate immune cell response. We could detect more CD11b⁺ myeloid cells, natural killer (NK) cells but not NK T cells in the liver of the mice receiving Akt-CTLs at day 7 after adoptive transfer (FIGS. 13B-D). The mice receiving Akt1-OT-I CTLs showed elevated ALT levels at day 7 and day 14 after the adoptive transfer of T cells and also cleared viruses at day 7 (FIGS. 12B and 12C). The mice receiving control OT-I CTLs did not show ALT elevation nor viral clearance after the adoptive transfer (FIGS. 12B and 12C). At day 60 after adoptive transfer, the mice were re-challenged by HDI of pENTRY-OL or pENTRY vector as HDI control to examine whether they developed antigen-specific T-cell memory. The mice receiving Akt1-CTLs showed mild liver damage as revealed by the ALT elevation during day 4 to day 7 after re-challenge. The ALT level in these mice was much less than that in their primary response (FIG. 12B). The mice receiving Akt1-OT-I CTLs re-expressed antigen as revealed by luciferase activity at day 61 and rapidly eliminated antigen within 3 days whereas the mice receiving ctrl-OT-I CTLs could not eliminate antigen after re-challenge (FIG. 12C).

Similar result was observed in the mice receiving Akt2-CTLs (FIGS. 13E and 13F). We could detect antigen-specific T-cell expansion in the liver of mice receiving Akt1-CTLs at day 7 after re-challenge (FIG. 12D). The liver histological examination showed that both the mice receiving ctrl- and Akt1-CTLs, respectively, had no obvious inflammation in the liver of mice after re-challenge (FIG. 12E). However, we could detect more CD8⁺ T cells as well as Gr-1⁺ myeloid cells in the liver sinusoid of the mice receiving Akt1-CTLs after re-challenge (FIGS. 12F and 12G). These data suggest the Akt-engineered CTLs don't only harbor strong effector functions but also are more efficient to develop T-cell memory and could eliminate antigen rapidly when re-encounter the antigen. During primary and re-call responses, we observed the recruitment of innate immune cells to the liver, which may be a reflection of tissue damage and for the purpose of tissue repair. It is also possible that the Gr-1⁺ myeloid cells contribute to the expansion of CTL population during the primary and re-call responses.

### Example 8: Akt Signaling in CTLs Enhances their Cytotoxic Function and Facilitates Tumor Killing

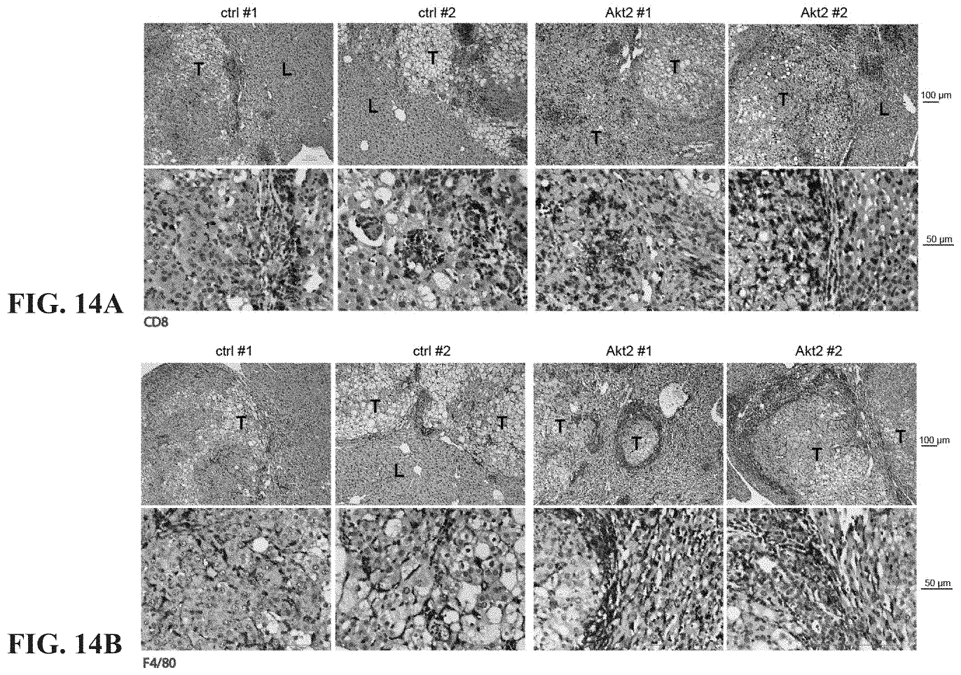

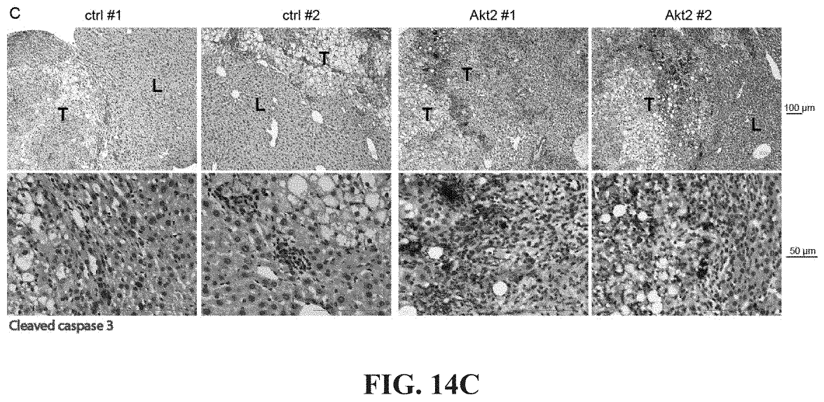

The capability of Akt-engineered CTLs in killing of hepatocellular carcinoma (HCC) is further examined and demonstrated that the tumor antigen-specific Akt2-engrafted CD8⁺ CTLs can accumulate in the tumor sites as well as in the liver at day 10 after adoptive transfer into HCC-bearing mice (FIG. 14A). These Akt2-CTLs change the tumor microenvironment and attract or activate the surrounding F4/80⁺ macrophages in tumor sites (FIG. 14B).

A lot of cleaved caspase 3-positive tumor cells are detected in the mice receiving Akt2-CTLs but not in ctrl mice (FIG. 14C). Serum ALT is elevated in the mice receiving Akt2-CTLs starting from day 3 after adoptive transfer but not in ctrl mice (118.1 U/L vs. 22.8 U/L). The level of ALT in mice receiving Akt2-CTLs is continuously increasing at least until day 10 after adoptive transfer (590.5 U/L).

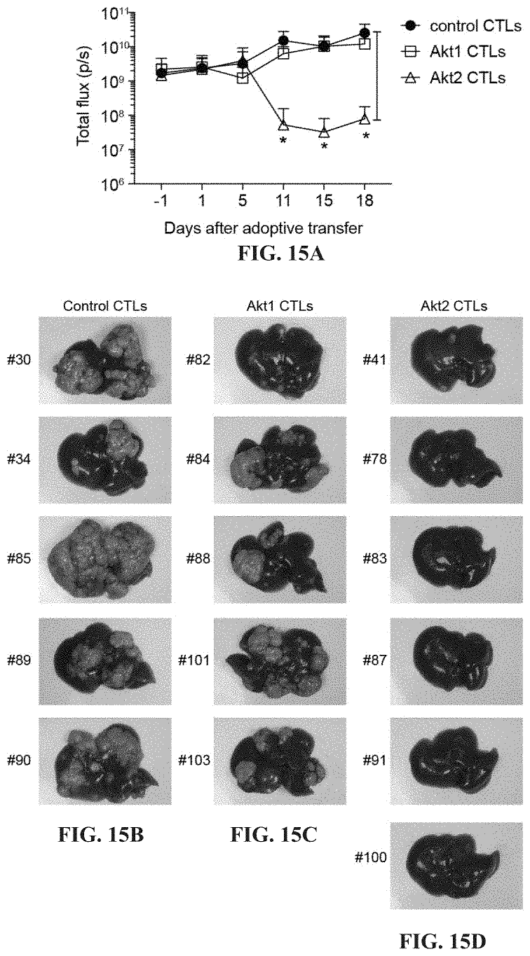

Ctrl-, Akt1- and Akt2-engineered HBc₉₃₋₁₀₀-specific CTLs are adoptively transferred into HCC-bearing mice, respectively. The oncogenes-induced HCC mouse model is engineered to express luciferase and surrogate tumor antigen-HBc₉₃₋₁₀₀ peptide in the tumor. The tumor growth can be monitored using IVIS and demonstrate that Akt2- but not ctrl- or Akt1-CTLs effectively eliminate HCC as shown by the reduction of in vivo bioluminescence and the disappearance of tumor nodules in the livers of mice receiving Akt2-CTLs (FIGS. 15A-D).

It can be concluded that Akt2 activation enables CTLs to have strong effector functions to kill tumor cells in the liver.

### Example 9: Anti-Tumor Capability of Akt-Engineered Chimeric Antigen Receptor (CAR) T Cells

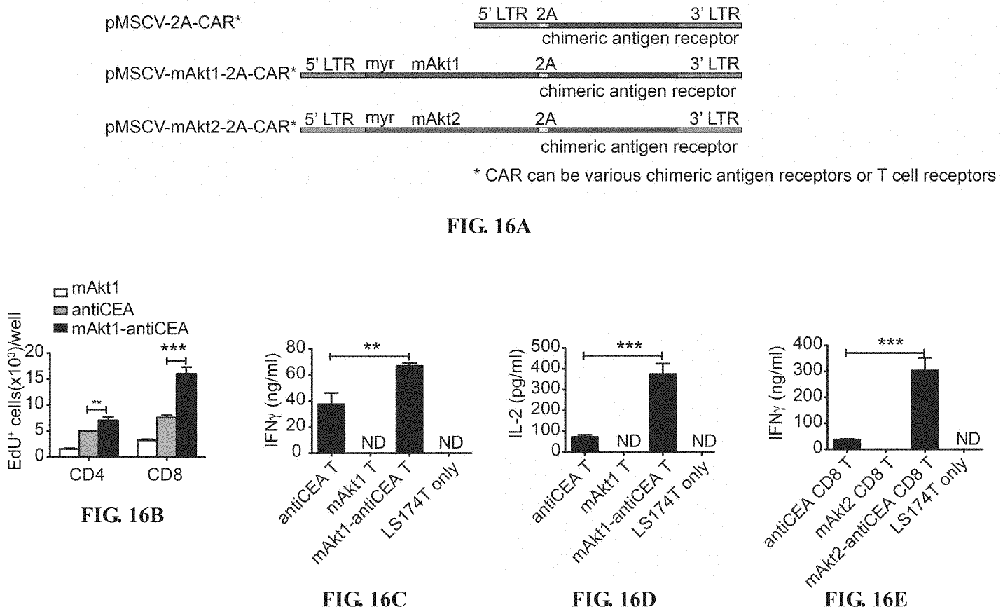

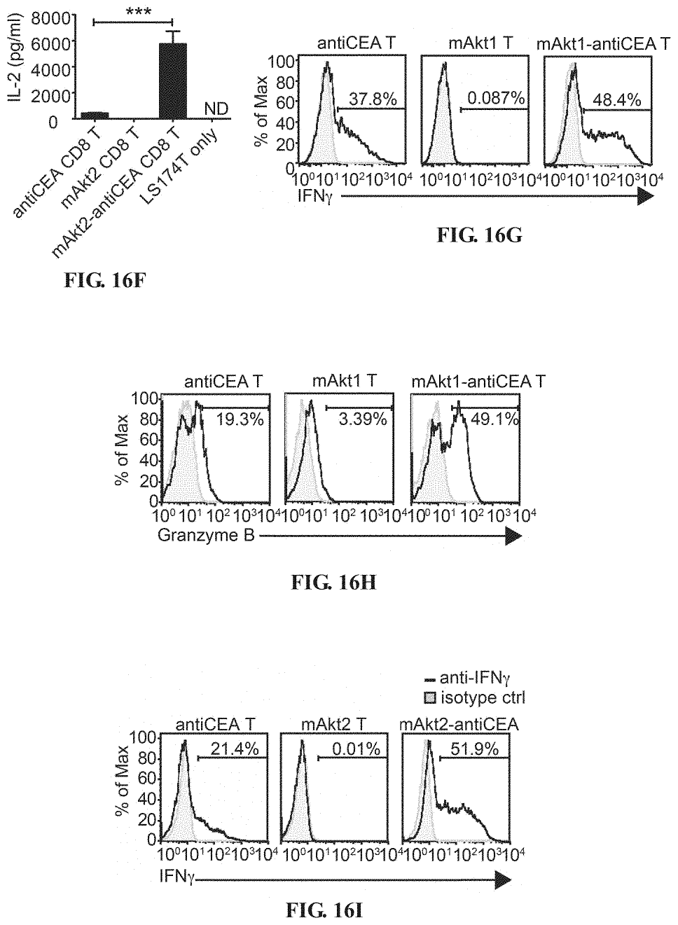

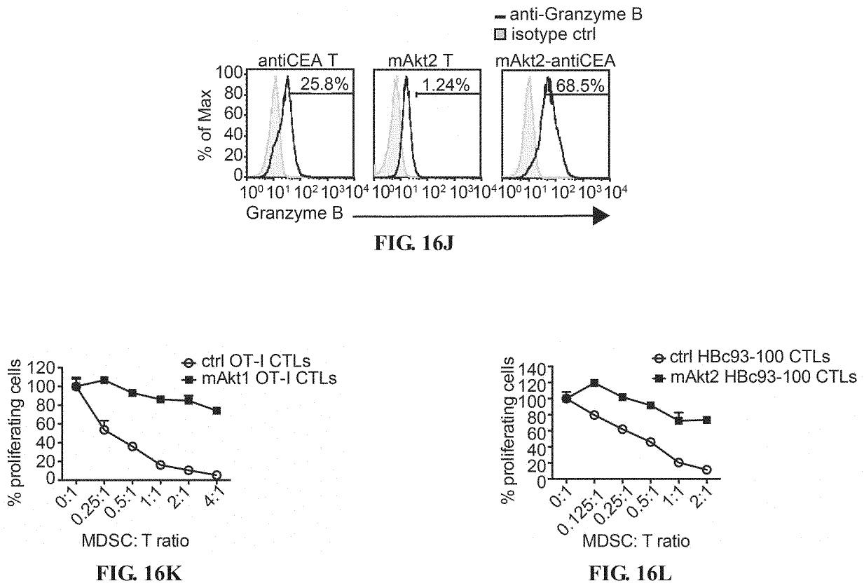

To further explore the potential application of Akt molecules on cancer immunotherapy, plasmids carrying human or mouse Akt1 or Akt2 genes are constructed and the ORF encoding anti-CEA chimeric antigen receptor (CAR) (FIG. 16A). The construction of the recombinant anti-CEA chimeric antigen receptor used in this present invention were described in Hombach et al. (Hombach, A.; Wieczarkowiecz, A.; Marquardt, T.; Heuser, C.; Usai, L.; Pohl, C.; Seliger, B.; Abken, H., Tumor-specific T cell activation by recombinant immunoreceptors: CD3ζ signaling and CD28 costimulation are simultaneously required for efficient IL-2 secretion and can be integrated into one combined CD28/CD3 signaling receptor molecule. J Immunol 2001, 167 (11), 6123-31). Activated mouse CD3⁺ T cells are modified by recombinant retroviruses carrying mouse AKT1 gene, anti-CEA CAR ORF or both and then are monitored for their proliferation capability, cytokine production and cytotoxicity.

The modified CTLs are co-cultured with a colorectal adenocarcinoma cell line with the expression of CEA, LS174T and the proliferation of the CTLs is monitored through detection of incorporation of a thymidine analog, EdU. Both CD4⁺ and CD8⁺ T cells with the engraftment of anti-CEA CAR can respond to stimulation of LS174T and proliferate. Akt signaling further enhances the proliferative capability of anti-CEA CAR-engrafted CD4⁺ and CD8⁺ T cells (FIG. 16B).

Higher levels of IL-2 and IFNγ are detected in the culture medium of co-culture of LS174T cell line with T cells expressing anti-CEA CAR and Akt1 or Akt2 molecules compared that of T cells expressing solely anti-CEA CAR (FIGS. 16C-F). Intracellular staining of IFNγ and granzyme B of the CD8⁺ T cells co-cultured with LS174T cells also proves that Akt1 or Akt2 overexpression can enhance the cytokine production and cytotoxicity in CTLs (FIGS. 16G-J).

Akt1-overexpressing and Akt2-overexpressing CTLs are shown to have the capability to overcome the proliferative arrest induced by myeloid-derived suppressor cells (MDSCs) (FIGS. 16K and 16L), which strongly suggests that the potential application of Akt molecules on T-cell engineering technology e.g. CAR T cells for immunotherapy.

This present invention provides a method able to enhance survival and functionality of anti-tumor or anti-viral T cells through overexpression of Akt molecules in CTLs. The Akt-overexpressing CTLs are shown to have high proliferation capability and superior effector functions during encounter with the antigen in the liver, which suggests that the Akt molecules can help the CTLs to overcome T-cell exhaustion in the inhibitory microenvironment. This present invention further shows expression of Akt molecules can facilitate anti-viral and anti-tumor CTL responses e.g. proliferation, cytokine production and cytotoxicity. Moreover, it enables the CTLs resistance to proliferative arrest induced by MDSCs. To sum up, the expression of constitutively active Akt molecules enable T cells to gain the privilege to survive and to kill in the tolerogenic liver or tumor microenvironments. The active Akt molecules only when in combination with TCR signaling can trigger massive proliferative response of CTLs and therefore are safe to be applied to T-cell engineering of CTLs. Inventors therefore have the following claims for the compositions comprising the anti-tumor or anti-viral engineered T cells and the methods using thereof for treatment of chronic viral infections and malignancies.

1. A composition for reducing immune tolerance which comprising an engineered cell overexpressing Akt molecules, the engineered cell is engineered with a polynucleotide encoding:
a. an Akt isoform; and
b. a peptide leading the Akt isoform to cell membrane of the engineered cell.

2. The composition according to claim 1, wherein the Akt isoform is selected from the group consisting of Akt1, Akt2, and Akt3, or a combination thereof.

3. The composition according to claim 1, wherein the peptide is a myristoylation-targeting sequence set forth in SEQ ID NO: 7.

4. The composition according to claim 1, wherein the polynucleotide further comprising a fragment encoding a chimeric antigen receptor or a recombinant T cell receptor.

5. The composition according to claim 4, wherein the polynucleotide further comprising a fragment encoding a linker between the Akt isoform and the chimeric antigen receptor or the recombinant T cell receptor.

6. The composition according to claim 5, wherein the linker is a 2A peptide set forth in SEQ ID NO: 9.

7. The composition according to claim 1, wherein the engineered cell is a T cell, a nature killer cell, a hematopoietic stem cell, an embryonic stem cell or a pluripotent stem cell.

8. A method for treating a virus infection disease in a subject comprising administering to the subject an effective amount of the composition according to claim 1.

9. The method according to claim 8, wherein the virus infection disease is hepatitis.

10. A method for treating a cancer in a subject comprising administering to the subject an effective amount of the composition according to claim 1.

11. The method according to claim 10, wherein the cancer is a liver cancer.

12. The method according to claim 11, wherein the liver cancer comprising hepatocellular carcinoma, bile duct carcinoma, hepatic angiosarcoma and epithelioid hemangioendothelioma.

13. A method for treating a cancer in a subject comprising administering to the subject an effective amount of the composition according to claim 4.

14. A method for producing the composition according to claim 1, which comprising transferring a recombinant viral or transposon vector into a target cell, and expanding the target cell.

15. The method according to claim 14, wherein the recombinant viral or transposon vector can be a retrovirus, a lentivirus, an adenovirus, an adeno-associated virus, or other related viruses and various transposon systems can be used in transduction or integration of transgenes.

16. The method according to claim 14, wherein the recombinant viral or transposon vector can be amplified through plasmid amplification, in vitro transcription or in vitro synthesis and transfected into the target cell through electroporation, liposome or other chemical vehicles.

17. The method according to claim 14, wherein the target cell can be a T cell, a nature killer cell, a hematopoietic stem cell, an embryonic stem cell or a pluripotent stem cell.

18. The method according to claim 14, wherein the target cell can be further modified by viral transduction and DNA or RNA transfection.

19. The method according to claim 14, wherein expanding the target cell comprising stimulating the target cell with soluble, plate-bound anti-CD3 and anti-CD28 antibodies or with anti-CD3 and anti-CD28 beads with supplement of cytokines to enhance the growth of the target cell.


SEQUENCE LISTING (14 sequences)

SEQ ID NO: 1 - protein, 480 aa, Mus musculus
MNDVAIVKEGWLHKRGEYIKTWRPRYFLLKNDGTFIGYKERPQDVDQRESPLNNFSVAQCQLMKTERPRPNTFIIRCLQWTTVIERTFHVETPEEREEWATAIQTVADGLKRQEEETMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPTQRLGGGSEDAKEIMQHRFFANIVWQDVYEKKLSPPFKPQVTSETDTRYFDEEFTAQMITITPPDQDDSMECVDSERRPHFPQFSYSASGTA

SEQ ID NO: 2 - DNA, 1440 bp, Mus musculus
atgaacgacgtagccattgtgaaggagggctggctgcacaaacgaggggaatatattaaaacctggcggccacgctacttcctcctcaagaacgatggcacctttattggctacaaggaacggcctcaggatgtggatcagcgagagtccccactcaacaacttctcagtggcacaatgccagctgatgaagacagagcggccaaggcccaacacctttatcatccgctgcctgcagtggaccacagtcattgagcgcaccttccatgtggaaacgcctgaggagcgggaagaatgggccaccgccattcagactgtggcagatggactcaagaggcaggaagaagagacgatggacttccgatcaggctcacccagtgacaactcaggggctgaagagatggaggtgtccctggccaagcccaagcaccgtgtgaccatgaacgagtttgagtacctgaagctactgggcaagggcacctttgggaaggtgattctggtgaaagagaaggccacaggccgctactatgccatgaagatcctcaagaaggaggtcatcgtcgccaaggatgaggttgcccacacgcttactgagaaccgtgtcctgcagaactctaggcatcccttccttacggccctcaagtactcattccagacccacgaccgcctctgctttgtcatggagtatgccaacgggggcgagctcttcttccacctgtctcgagagcgtgtgttctccgaggaccgggcccgcttctatggtgcggagattgtgtctgccctggactacttgcactccgagaagaacgtggtgtaccgggacctgaagctggagaacctcatgctggacaaggacgggcacatcaagataacggacttcgggctgtgcaaggaggggatcaaggacggtgccactatgaagacattctgcggaacgccggagtacctggcccctgaggtgctggaggacaacgactacggccgtgcagtggactggtgggggctgggcgtggtcatgtacgagatgatgtgtggccgcctgcccttctacaaccaggaccacgagaagctgttcgagctgatcctcatggaggagatccgcttcccgcgcacactcggccctgaggccaagtccctgctctccgggctgctcaagaaggaccctacacagaggctcggtgggggctccgaggatgccaaggagatcatgcagcaccggttctttgccaacatcgtgtggcaggatgtgtatgagaagaagctgagcccacctttcaagccccaggtcacctctgagactgacaccaggtatttcgatgaggagttcacagctcagatgatcaccatcacgccgcctgatcaagatgacagcatggagtgtgtggacagtgagcggaggccgcacttcccccagttctcctactcagccagtggcacagcc

SEQ ID NO: 3 - protein, 481 aa, Mus musculus
MNEVSVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSLKQRGPGEDAMDYKCGSPSDSSTSEMMEVAVNKARAKVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEDRARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLGPEAKSLLAGLLKKDPKQRLGGGPSDAKEVMEHRFFLSINWQDVVQKKLLPPFKPQVTSEVDTRYFDDEFTAQSITITPPDRYDSLDPLELDQRTHFPQFSYSASIRE

SEQ ID NO: 4 - DNA, 1443 bp, Mus musculus
atgaatgaggtatctgtcatcaaagaaggctggctccacaaacgtggtgaatacatcaagacctggaggccacggtacttccttctgaagagtgatggatctttcattgggtataaggagaggcccgaggcccctgaccagaccttaccccccctgaacaatttctctgtagcagaatgccagctgatgaagactgagaggccacgacccaacacctttgtcatacgctgcctgcagtggaccacagtcatcgagaggaccttccatgtagactctccagatgagagggaagagtggatgcgggctatccagatggtcgccaacagtctgaagcagcggggcccaggtgaggacgccatggattacaagtgtggctcccccagtgactcttccacatctgagatgatggaggtagctgtcaacaaggcacgggccaaagtgaccatgaatgacttcgattatctcaaactcctcggcaagggcaccttcggcaaggtcattctggttcgagagaaggccactggccgctattatgccatgaagatcctgcgcaaggaggtcatcattgcaaaggatgaagtcgcccacacagtcacagagagccgggttctgcagaataccaggcaccccttccttacagccctcaagtatgccttccagacccatgaccgcctatgctttgtgatggagtatgccaacgggggtgagctgtttttccacctctctcgggagcgagtcttcacggaggatcgggcgcgcttttatggagcagagattgtgtcagctctggagtatttgcactcgagagatgtggtgtaccgtgacatcaagctggaaaaccttatgttggacaaagatggccacatcaagatcactgactttggcttgtgcaaagagggcatcagtgatggagccaccatgaaaaccttctgtggtaccccggagtacttggcgcctgaggtgctagaggacaatgactatgggcgagcagtggactggtgggggctgggtgtggtcatgtatgagatgatgtgtggccgcctgccattctacaaccaggaccacgagcgcctctttgagctcattcttatggaggagatccgcttcccgcgcacactcgggccagaggccaagtccctgctggctggactgctgaagaaggacccaaagcagaggctcggcggaggtcccagtgatgcgaaggaggtcatggagcatagattcttcctcagcatcaactggcaggacgtggtacagaaaaagctcctgccacccttcaaacctcaggtcacttcagaagtggacacaaggtactttgatgacgagttcaccgcccagtccatcacaatcacacccccagaccgatatgacagcctggacccgctggaactggaccagcggacgcacttcccccagttctcctactcagccagcatccgagag

SEQ ID NO: 5 - protein, 474 aa, Mus musculus
MSDVTIVKEGWVQKRGEYIKNWRPRYFLLKTDGSFIGYKEKPQDVDLPYPLNNFSVAKCQLMKTERPKPNTFIIRCLQWTTVIERTFHVDTPEEREEWTEAIQAVADRLQRQEEERMNCSPTSQIDNIGEEEMDASTTHHKRKTMNDFDYLKLLGKGTFGKVILVREKASGKYYAMKILKKEVIIAKDEVAHTLTESRVLKNTRHPFLTSLKYSFQTKDRLCFVMEYVNGGELFFHLSRERVFSEDRTRFYGAEIVSALDYLHSGKIVYRDLKLENLMLDKDGHIKITDFGLCKEGITDAATMKTFCGTPEYLAPEVLEDNDYGRAVDWWGLGVVMYEMMCGRLPFYNQDHEKLFELILMEDIKFPRTLSSDAKSLLSGLLIKDPNKRLGGGPDDAKEIMRHSFFSGVNWQDVYDKKLVPPFKPQVTSETDTRYFDEEFTAQTITITPPEKYDDDGMDGMDNERRPHFPQFSYS

SEQ ID NO: 6 - DNA, 1437 bp, Mus musculus
atgagcgatgttaccattgtgaaagaaggttgggttcagaagaggggagaatatataaaaaactggaggccaagatacttccttttgaagacagatggctcattcataggctataaggagaaacctcaagatgtggacttaccttatcccctcaacaacttctcagtggcaaaatgtcagttaatgaaaacagaacgaccaaagccaaatacatttattatcagatgtcttcagtggaccactgttatagagagaacatttcatgtagatacaccagaggaaagagaagagtggacggaagctatccaagccgtagccgaccgattgcagaggcaagaggaggagaggatgaattgtagcccaacctcacagattgataatataggagaagaagagatggatgcgtctacaacccatcataaaagaaagacgatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactcttactgaaagcagagtactaaagaacaccagacatccatttttaacatccttgaaatattccttccagacaaaagaccgtttgtgttttgtgatggaatatgttaatggcggagagctgtttttccatttgtcgagagagcgagtgttctctgaggaccgcacacgtttctatggtgcagaaattgtctctgctttggactatctacattctggaaagattgtgtaccgtgatctcaagttggagaatttgatgctagataaggatggccatataaaaattacggattttgggctttgcaaagaagggatcacagatgcagctaccatgaagacattctgtggcacaccagagtacctggcaccagaggtattagaagataatgactatggccgagccgtggactggtggggcttaggtgttgtcatgtatgaaatgatgtgtggaaggttgcctttctacaaccaggatcatgagaaactctttgaattaatactaatggaagacattaaattcccccgaacactctcttcagatgcaaaatcattgctttcagggctcttgataaaggatccaaataaacgccttggtggagggccagatgatgcaaaagaaatcatgaggcatagttttttttctggagtaaactggcaagatgtatatgacaaaaagcttgtacctccttttaagcctcaagtaacatctgaaacagacacccgatattttgatgaagaatttacagctcagactattacaataacaccacctgaaaagtatgacgacgacggcatggacggcatggacaacgagcggcggccacacttccctcagttctcctactctgcaagcggacgggaa

SEQ ID NO: 7 - protein, 15 aa, Artificial Sequence
  Synthetic
MGSSKSKPKDPSQRA

SEQ ID NO: 8 - DNA, 45 bp, Artificial Sequence
  Synthetic
atggggagcagcaagagcaagcccaaggaccccagccagcgcgcc

SEQ ID NO: 9 - protein, 22 aa, Artificial Sequence
  Synthetic
GSGATNFSLLKQAGDVEENPGP

SEQ ID NO: 10 - DNA, 66 bp, Artificial Sequence
  Synthetic
ggaagcggagctactaacttcagcctgctgaagcaggctggagacgtggaggagaaccctggacct

SEQ ID NO: 11 - protein, 480 aa, Homo sapiens [sequence omitted]

SEQ ID NO: 12 - DNA, 1440 bp, Homo sapiens
atgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcc

SEQ ID NO: 13 - protein, 481 aa, Homo sapiens
MNEVSVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSLKQRAPGEDPMDYKCGSPSDSSTTEEMEVAVSKARAKVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLGLLELDQRTHFPQFSYSASIRE

SEQ ID NO: 14 - DNA, 1443 bp, Homo sapiens
atgaatgaggtgtctgtcatcaaagaaggctggctccacaagcgtggtgaatacatcaagacctggaggccacggtacttcctgctgaagagcgacggctccttcattgggtacaaggagaggcccgaggcccctgatcagactctaccccccttaaacaacttctccgtagcagaatgccagctgatgaagaccgagaggccgcgacccaacacctttgtcatacgctgcctgcagtggaccacagtcatcgagaggaccttccacgtggattctccagacgagagggaggagtggatgcgggccatccagatggtcgccaacagcctcaagcagcgggccccaggcgaggaccccatggactacaagtgtggctcccccagtgactcctccacgactgaggagatggaagtggcggtcagcaaggcacgggctaaagtgaccatgaatgacttcgactatctcaaactccttggcaagggaacctttggcaaagtcatcctggtgcgggagaaggccactggccgctactacgccatgaagatcctgcggaaggaagtcatcattgccaaggatgaagtcgctcacacagtcaccgagagccgggtcctccagaacaccaggcacccgttcctcactgcgctgaagtatgccttccagacccacgaccgcctgtgctttgtgatggagtatgccaacgggggtgagctgttcttccacctgtcccgggagcgtgtcttcacagaggagcgggcccggttttatggtgcagagattgtctcggctcttgagtacttgcactcgcgggacgtggtataccgcgacatcaagctggaaaacctcatgctggacaaagatggccacatcaagatcactgactttggcctctgcaaagagggcatcagtgacggggccaccatgaaaaccttctgtgggaccccggagtacctggcgcctgaggtgctggaggacaatgactatggccgggccgtggactggtgggggctgggtgtggtcatgtacgagatgatgtgcggccgcctgcccttctacaaccaggaccacgagcgcctcttcgagctcatcctcatggaagagatccgcttcccgcgcacgctcagccccgaggccaagtccctgcttgctgggctgcttaagaaggaccccaagcagaggcttggtggggggcccagcgatgccaaggaggtcatggagcacaggttcttcctcagcatcaactggcaggacgtggtccagaagaagctcctgccacccttcaaacctcaggtcacgtccgaggtcgacacaaggtacttcgatgatgaatttaccgcccagtccatcacaatcacaccccctgaccgctatgacagcctgggcttactggagctggaccagcggacccacttcccccagttctcctactcggccagcatccgcgag